(19) United States
(12) Patent Application Publication
(10) Pub. No.: US 2020/0345976 A1
(43) Pub. Date: Nov. 5, 2020

(54) Drug Delivery Devices and Methods for Use with a Urinary Catheter

(71) Applicant: TARIS BIOMEDICAL LLC, Lexington, MA (US)

(72) Inventors: Joseph Kalt, Arlington, MA (US); Heejin Lee, Bedford, MA (US)

(21) Appl. No.: 16/760,729

(22) PCT Filed: Nov. 8, 2018
(86) PCT No.: PCT/US2018/059825

§ 371 Date: Apr. 30, 2020

Related U.S. Application Data
(60) Provisional application No. 62/584,006, filed on Nov. 9, 2017.

(51) Int. Cl. A61M 25/00; A61M 25/10

(52) U.S. Cl. CPC A61M 25/0017; A61M 25/10; A61M 2210/1085; A61M 2202/048; A61M 2025/105; A61M 2202/064; A61M 2210/1089

(57) ABSTRACT

Drug delivery devices (200) and methods of their use are provided. In one embodiment, a drug delivery device (200) for use with a urinary catheter (100) includes a drug reservoir configured to be disposed outside of a patients body, and a flexible elongate body (220) attached to the drug reservoir (230) and configured to traverse the patients urethra to reach the bladder. The drug reservoir (230) includes a drug chamber (234) containing a drug therein, a fluid chamber (236) containing a fluid therein, and an osmotic barrier (238) separating the drug chamber (234) and the fluid chamber (236). The body includes a drug delivery lumen (222) extending therethrough and in fluid communication with the drug chamber (234).

### CROSS-REFERENCE TO RELATED APPLICATIONS

This application claims priority benefit to U.S. Provisional Patent Application No. 62/584,006, filed Nov. 9, 2017, which is incorporated herein by reference.

### TECHNICAL FIELD

This application relates generally to medical devices and methods, and more particularly to drug delivery devices and related methods of using such devices for controlled delivery of a drug to a selected region of the lower urinary tract, such as the bladder, of a catheterized patient.

### BACKGROUND

Various types of drug delivery devices and methods have been developed for delivering a drug to the lower urinary tract of a patient. For example, certain drug delivery devices may be implanted and retained in the patient's urinary bladder and configured to controllably release a drug therein over an extended period of time to treat a number of conditions. However, use of such drug delivery devices may not be practical, or even possible, when the patient is catheterized. Urinary catheters, such as Foley catheters, are often used in both acute (e.g., post-surgical) and chronic (e.g., spinal cord injury) settings to maintain continuous urethral patency. Because urinary catheters allow urine to drain freely from the bladder and thus keep the bladder continuously empty (aside from minimal residual urine), use of intravesical drug delivery devices which benefit from or require the presence of a substantial amount of urine in the bladder may not be optimal or feasible for catheterized patients. Additionally, a distal end portion of the urinary catheter residing in the bladder may interfere with desired interaction between the drug delivery device and the bladder and/or may inhibit desired movement of the drug delivery device within the bladder. Furthermore, the presence of the intravesical drug delivery device within the bladder, along with the distal end portion of the urinary catheter, may interfere with the desired function of the catheter and/or may result in issues of patient tolerability.

It therefore would be desirable to provide new and improved drug delivery devices and methods for controlled delivery of a drug to a selected region of the lower urinary tract, such as the bladder, of a catheterized patient. Such drug delivery devices should be configured to be easily inserted into and removed from the bladder, either along with or separately from a urinary catheter, such as a Foley catheter. It would be advantageous for such devices to include a sufficiently large drug payload in order to provide drug delivery over an extended period of time, without interfering with the desired function of the urinary catheter and without occupying such a significant portion of the bladder that would result in patient tolerability issues. It also would be advantageous for such devices and methods to prevent or inhibit microbial infections that otherwise may develop from continued catheterization.

### BRIEF SUMMARY

Drug delivery devices, systems, and methods for controlled delivery of a drug to a selected region of the lower urinary tract, such as the bladder, of a catheterized patient are provided. According to one aspect, a drug delivery device for use with a urinary catheter is provided. In one embodiment, the drug delivery device includes a drug reservoir configured to be disposed outside of a patient's body, and a flexible elongate body attached to the drug reservoir and configured to traverse the patient's urethra to reach the bladder. The drug reservoir includes a drug chamber containing a drug therein, a fluid chamber containing a fluid therein, and an osmotic barrier separating the drug chamber and the fluid chamber. The body includes a drug delivery lumen extending therethrough and in fluid communication with the drug chamber.

In another aspect, a urinary catheter and drug delivery system is provided. In one embodiment, the system includes (i) a urinary catheter configured to allow urine to drain from a patient's bladder, and (ii) a drug delivery device configured for use with the urinary catheter. The urinary catheter includes a flexible elongate catheter body configured to traverse the patient's urethra to reach the bladder, and the catheter body includes a drainage lumen extending therethrough. The drug delivery device includes a drug reservoir configured to be disposed outside of the patient's body, and a flexible elongate device body attached to the drug reservoir and configured to traverse the patient's urethra to reach the bladder. The drug reservoir includes a drug chamber containing a drug therein, a fluid chamber containing a fluid therein, and an osmotic barrier separating the drug chamber and the fluid chamber. The body includes a drug delivery lumen extending therethrough and in fluid communication with the drug chamber.

In still another aspect, a method of administering a drug to a patient in need thereof is provided. In one embodiment, the method includes inserting distal end portions of a drug delivery device and a urinary catheter through the patient's urethra and positioning the distal end portions within the bladder, while maintaining proximal end portions of the drug delivery device and the urinary catheter positioned outside of the patient's body; allowing urine to drain from the bladder through the urinary catheter; and delivering a drug, via osmotic pressure, from the proximal end portion of the drug delivery device into the bladder.

In another aspect, a urinary catheter and drug delivery system is provided. In one embodiment, the system includes (i) a urinary catheter configured to allow urine to drain from a patient's bladder, and (ii) a drug delivery device attached to the urinary catheter. The urinary catheter includes a flexible elongate catheter body configured to traverse the patient's urethra to reach the bladder, and the catheter body includes a drainage lumen extending therethrough from a distal opening to proximal opening defined in the catheter body. The drug delivery device includes a drug reservoir positioned near the distal opening of the drainage lumen and configured to be disposed within the patient's bladder, and the drug reservoir includes a drug chamber containing a drug therein.

In still another aspect, a method of administering a drug to a patient in need thereof is provided. In one embodiment, the method includes inserting a drug delivery device and a distal end portion of a urinary catheter through the patient's urethra and positioning the drug delivery device and the distal end portion of the urinary catheter within the bladder, wherein the urinary catheter includes a flexible elongate catheter body including a drainage lumen extending therethrough from a distal opening to proximal opening defined in the catheter body, and wherein the drug delivery device includes a drug reservoir positioned near the distal opening of the drainage lumen and including a drug chamber containing a drug therein; allowing urine to drain from the bladder through the drainage lumen; and delivering the drug from the drug chamber into the bladder.

These and other aspects and embodiments of the present disclosure will be apparent or will become apparent to one of ordinary skill in the art upon review of the following detailed description when taken in conjunction with the drawings and the appended claims.

### BRIEF DESCRIPTION OF THE DRAWINGS

The detailed description is set forth with reference to the accompanying drawings. The use of the same reference numerals may indicate similar or identical items. Various embodiments of the disclosure may utilize components and/or features other than those illustrated in the drawings, and the illustrated components and/or features may not be present in various embodiments. Components and/or features illustrated in the drawings are not necessarily drawn to scale. In some figures, the relative size of certain components and/or features may be exaggerated for ease of illustration. Throughout this disclosure, depending on context, singular and plural terminology may be used interchangeably.

FIG. 1A is a plan view of a urinary catheter in accordance with one or more embodiments of the disclosure, showing a balloon of the catheter in a collapsed configuration.

FIG. 1B is a plan view of the urinary catheter of FIG. 1A, showing the balloon in an expanded configuration.

FIG. 1C is a detailed plan view of a distal end portion of the urinary catheter of FIG. 1A, showing the balloon in the collapsed configuration.

FIG. 1D is a detailed plan view of the distal end portion of the urinary catheter of FIG. 1A, showing the balloon in the expanded configuration.

FIG. 1E is a plan view of the urinary catheter of FIG. 1A positioned partially within a patient, showing the distal end portion of the urinary catheter positioned within the patient's bladder and a proximal end portion of the urinary catheter positioned outside of the patient's body.

FIG. 2A is a plan view of a drug delivery device in accordance with one or more embodiments of the disclosure, which may be used with the urinary catheter of FIG. 1A.

FIG. 2B is a side view of the drug delivery device of FIG. 2A.

FIG. 2C is a cross-sectional top view of the drug delivery device of FIG. 2A, taken along line 2C-2C in FIG. 2B.

FIG. 2D is a cross-sectional top view of the drug delivery device of FIG. 2A, taken along line 2D-2D in FIG. 2B.

FIG. 3A is a plan view of a urinary catheter and drug delivery system in accordance with one or more embodiments of the disclosure including the urinary catheter of FIG. 1A and the drug delivery device of FIG. 2A, showing the balloon of the catheter in the collapsed configuration.

FIG. 3B is a plan view of the urinary catheter and drug delivery system of FIG. 3A, showing the balloon of the catheter in the expanded configuration.

FIG. 3C is a detailed plan view of a distal end portion of the urinary catheter and drug delivery system of FIG. 3A, showing the balloon of the catheter in the collapsed configuration.

FIG. 3D is a detailed plan view of a distal end portion of the urinary catheter and drug delivery system of FIG. 3A, showing the balloon of the catheter in the expanded configuration.

FIG. 3E is a plan view of the urinary catheter and drug delivery system of FIG. 3A positioned partially within a patient, showing the distal end portion of the system positioned within the patient's bladder and a proximal end portion of the system positioned outside of the patient's body.

FIG. 3F is a detailed plan view of a distal end portion of a urinary catheter and drug delivery system in accordance with one or more embodiments of the disclosure including the urinary catheter of FIG. 1A and the drug delivery device of FIG. 2A, showing the balloon of the catheter in the collapsed configuration.

FIG. 3G is a detailed plan view of the distal end portion of the urinary catheter and drug delivery system of FIG. 3F, showing the balloon of the catheter in the expanded configuration.

FIG. 3H is a detailed plan view of a distal end portion of a urinary catheter and drug delivery system in accordance with one or more embodiments of the disclosure including the urinary catheter of FIG. 1A and the drug delivery device of FIG. 2A, showing the balloon of the catheter in the collapsed configuration.

FIG. 3I is a detailed plan view of the distal end portion of the urinary catheter and drug delivery system of FIG. 3H, showing the balloon of the catheter in the expanded configuration.

FIG. 3J is a detailed plan view of a distal end portion of a urinary catheter and drug delivery system in accordance with one or more embodiments of the disclosure including the urinary catheter of FIG. 1A and the drug delivery device of FIG. 2A, showing the balloon of the catheter in the collapsed configuration.

FIG. 3K is a detailed plan view of the distal end portion of the urinary catheter and drug delivery system of FIG. 3J, showing the balloon of the catheter in the expanded configuration.

FIG. 4A is a plan view of a urinary catheter and drug delivery system in accordance with one or more embodiments of the disclosure including the urinary catheter of FIG. 1A and a drug delivery device, showing the balloon of the catheter in the collapsed configuration.

FIG. 4B is a plan view of the urinary catheter and drug delivery system of FIG. 4A, showing the balloon of the catheter in the expanded configuration.

FIG. 4C is a detailed plan view of a distal end portion of the urinary catheter and drug delivery system of FIG. 4A, showing the balloon of the catheter in the collapsed configuration.

FIG. 4D is a detailed cross-sectional plan view of a distal end portion of the urinary catheter and drug delivery system of FIG. 4A, showing the balloon of the catheter in the collapsed configuration.

FIG. 4E is a plan view of the urinary catheter and drug delivery system of FIG. 4A positioned partially within a patient, showing the distal end portion of the system positioned within the patient's bladder and a proximal end portion of the system positioned outside of the patient's body.

FIG. 5A is a plan view of a urinary catheter and drug delivery system in accordance with one or more embodiments of the disclosure including a urinary catheter and a drug delivery device, showing the balloon of the catheter in the collapsed configuration.

FIG. 5B is a plan view of the urinary catheter and drug delivery system of FIG. 5A, showing the balloon of the catheter in the expanded configuration.

FIG. 5C is a detailed plan view of a distal end portion of the urinary catheter and drug delivery system of FIG. 5A, showing the balloon of the catheter in the collapsed configuration.

FIG. 5D is a detailed cross-sectional plan view of a distal end portion of the urinary catheter and drug delivery system of FIG. 5A, showing the balloon of the catheter in the collapsed configuration.

FIG. 5E is a plan view of the urinary catheter and drug delivery system of FIG. 4A positioned partially within a patient, showing the distal end portion of the system positioned within the patient's bladder and a proximal end portion of the system positioned outside of the patient's body.

### DETAILED DESCRIPTION

Improved drug delivery devices, systems, and methods have been developed for controlled delivery of a drug to a selected region of the lower urinary tract, such as the bladder, of a catheterized patient. The drug delivery devices may be used with a urinary catheter, such as a Foley catheter, which collectively form a urinary catheter and drug delivery system. The drug delivery devices advantageously include a drug reservoir that resides outside of the patient's body during use of the device and is configured to operate as an osmotic pump to push a drug from the reservoir through a flexible elongate luminal body (e.g., a capillary tube) extending along or through the catheter for release of the drug into the bladder. The positioning of the drug reservoir outside of the patient's body advantageously allows the drug delivery device to include a sufficiently large drug payload for drug delivery over an extended period of time, while minimizing interference with the desired function of the urinary catheter and reducing the likelihood of patient tolerability issues. The drug delivery devices, systems, and methods may be used to controllably release a drug into the patient's bladder over an extended period of time to treat a number of bladder conditions, while also preventing or inhibiting microbial infections that otherwise may develop from continued catheterization.

The drug delivery device may be provided along with a urinary catheter, such as a Foley catheter, to collectively form a urinary catheter and drug delivery system. The system may be provided with the device and the catheter pre-assembled and permanently attached to one another, or the device and the catheter may be provided separately and configured for releasable attachment to one another. Alternatively, the drug delivery device may be configured for use in conjunction with a conventional urinary catheter, such as a commercially available Foley catheter.

As used herein, the term “patient” refers primarily to a human adult or child, but also may include other suitable mammalian animals, for example in a pre-clinical trial or in veterinary care.

Urinary Catheter

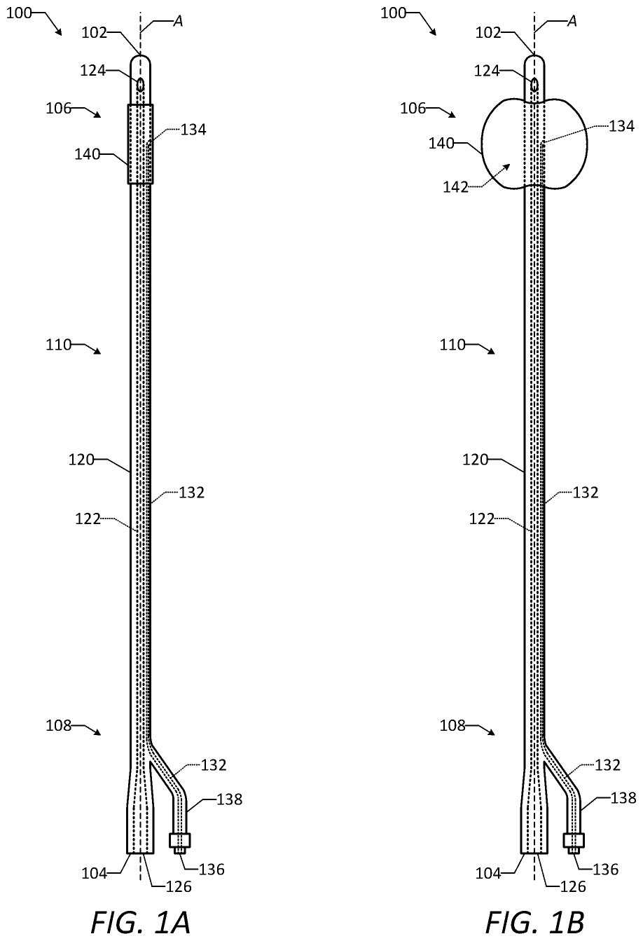

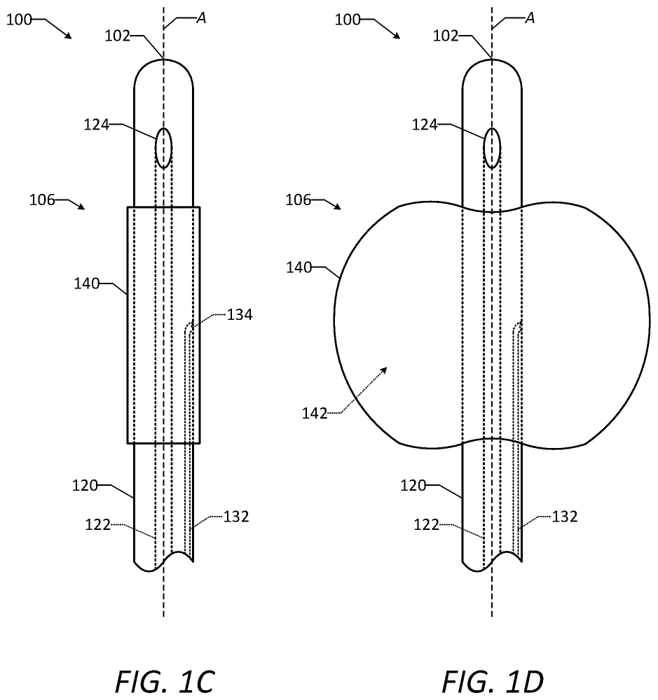

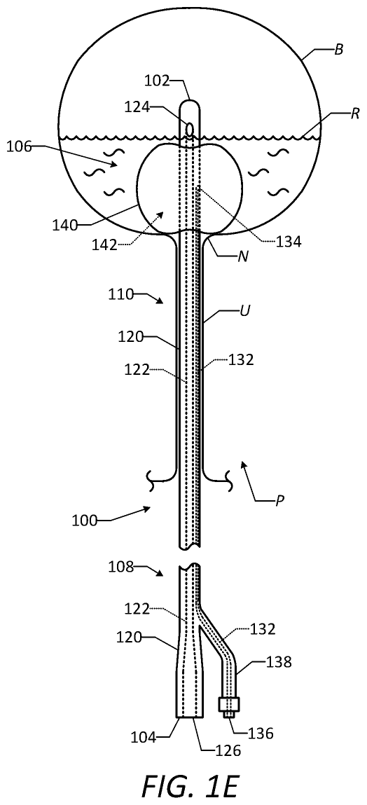

FIGS. 1A-1E illustrate a urinary catheter 100 (which also may be referred to as a “Foley catheter” or simply a “catheter”) configured to allow urine to drain freely from a patient's bladder, in accordance with one or more embodiments of the disclosure. During use, a portion of the catheter 100 may be inserted through the patient's urethra and into the bladder to maintain continuous urethral patency. Although the illustrated embodiment of the catheter 100 is configured as a Foley catheter, other types of catheters may be used in accordance with other embodiments of the disclosure. As described below, the urinary catheter 100 may be used with a drug delivery device 200 to form a urinary catheter and drug delivery system 300 which allows for continuous urine drainage in addition to controlled delivery of a drug to a selected region of the patient's lower urinary tract, such as the bladder, over an extended period of time.

As shown in FIG. 1A, the urinary catheter 100 has an elongated shape including a distal end 102 (which also may be referred to as a “bladder end”) and a proximal end 104 (which also may be referred to as an “external end”) positioned along a longitudinal axis A of the catheter 100. The urinary catheter 100 includes a distal end portion 106 (which also may be referred to as a “bladder end portion”) extending from the distal end 102 toward the proximal end 104 along the longitudinal axis A, a proximal end portion 108 (which also may be referred to as an “external end portion”) extending from the proximal end 104 toward the distal end 102 along the longitudinal axis A, and an intermediate portion 110 (which also may be referred to as a “urethral portion”) extending axially from the distal end portion 106 to the proximal end portion 108. When the catheter 100 is used to allow urine to drain from the patient's bladder, the distal end portion 106 may be inserted through the urethra and into the bladder, while the intermediate portion 110 resides within the urethra and the proximal end portion 108 resides outside of the patient's body.

The urinary catheter 100 includes a flexible elongate body 120 (which also may be referred to as a “catheter body” or a “catheter tube”) and an inflatable balloon 140 attached to the body 120, as shown. The body 120 may extend axially from the distal end 102 to the proximal end 104 of the catheter 100 and may be configured to traverse the patient's urethra to reach the bladder. As shown, the body 120 may have an elongated tubular shape and a circular cross-sectional shape, although other shapes of the body 120 may be used. As shown, a longitudinal axis of the body 120 may be coaxial with the longitudinal axis A of the catheter 100. The body 120 may include a drainage lumen 122 (which also may be referred to as a “primary lumen”) extending axially through the catheter 100 and configured to allow urine to flow therethrough from the bladder to a collection bag attached to the proximal end 104 of the catheter 100. In particular, the drainage lumen 122 may extend from a distal opening 124 (which also may be referred to as a “drainage entry opening”) defined in the body 120 to a proximal opening 126 (which also may be referred to as a “drainage exit opening”) defined in the body 120. As shown, the distal opening 124 may be defined in a sidewall of the body 120 and positioned near but spaced apart from the distal end 102 of the catheter 100, and the proximal opening 126 may be defined in or near the proximal end 104 of the catheter 100. In some embodiments, as shown, the drainage lumen 122 has a cylindrical shape and a circular axial cross-sectional shape, although other shapes of the drainage lumen 122 may be used. In some embodiments, as shown, a longitudinal axis of the drainage lumen 122 is coaxial with the longitudinal axis of the body 120 and the longitudinal axis A_{L} of the catheter 100.

The body 120 also may include an inflation lumen 132 (which also may be referred to as a “secondary lumen”) extending axially through the catheter 100 and configured to allow a fluid, such as sterile water, to be delivered therethrough from a fluid source attached to the proximal end 104 of the catheter 100 for inflation of the balloon 140. In particular, the inflation lumen 132 may extend from a distal opening 134 (which also may be referred to as a “inflation exit opening”) defined in the body 120 to a proximal opening 136 (which also may be referred to as a “inflation entry opening”) defined in the body 120. As shown, the distal opening 134 may be defined in a sidewall of the body 120 and spaced apart from the distal end 102 of the catheter 100, and the proximal opening 136 may be defined in or near the proximal end 104 of the catheter 100. For example, the proximal opening 136 may be defined in the proximal end of an inflation arm 138 of the body 120. In some embodiments, as shown, the inflation lumen 132 has a cylindrical shape and a circular axial cross-sectional shape, although other shapes of the inflation lumen 132 may be used. In some embodiments, as shown, a longitudinal axis of the inflation lumen 132 is offset from the longitudinal axis of the body 120 and the longitudinal axis A of the catheter 100.

The balloon 140 may be attached to body 120 and configured to be inflated from a collapsed configuration (which also may be referred to as a “deflated configuration”), as shown in FIG. 1A, to an expanded configuration (which also may be referred to as an “inflated configuration”), as shown in FIG. 1B. As shown, the balloon 140 may be positioned near but proximally spaced apart from the distal end 102 of the catheter 100. The balloon 140 also may be positioned near but proximally spaced apart from the distal opening 124 of the drainage lumen 122. The balloon 140 may be positioned over the distal opening 134 of the inflation lumen 132 and may include an internal cavity 142 in fluid communication with the inflation lumen 132. In this manner, the fluid may be passed through the inflation lumen 132 and fill the cavity 142 to inflate the balloon 140 from the collapsed configuration to the expanded configuration.

FIG. 1E illustrates use of the urinary catheter 100 to allow urine to drain from the bladder B of a patient P. With the balloon 140 in the collapsed configuration, the distal end portion 106 of the catheter 100 may be inserted through the urethra U and into the bladder B, such that the balloon 140 is disposed within the bladder B adjacent the bladder neck N, while the intermediate portion 110 of the catheter 100 is disposed within the urethra U and the proximal end portion 108 is disposed outside of the body of the patient P. Fluid, such as sterile water, then may be passed through the inflation lumen 132 and into the cavity 142 to inflate the balloon 140 to the expanded configuration, such that the balloon 140 forms a seal against the bladder neck N With the catheter 100 positioned as shown in FIG. 1E, urine may freely enter the distal opening 124 of the drainage lumen 122, pass through the drainage lumen 122, and be collected in a collection bag attached to the proximal end 104 of the catheter 100. As shown, some residual urine R may remain in the bladder B due to the position of the distal opening 124 of the drainage lumen 122 relative to the bladder neck N.

Although the urinary catheter 100 is shown and described as being a Foley catheter including the body 120 and the balloon 140, it will be appreciated that other configurations of the catheter 100, with or without a balloon, may be used according to various embodiments of the disclosure. Further, the catheter 100 may include other components and/or features in addition to those shown in the figures and described herein.

Drug Delivery Device

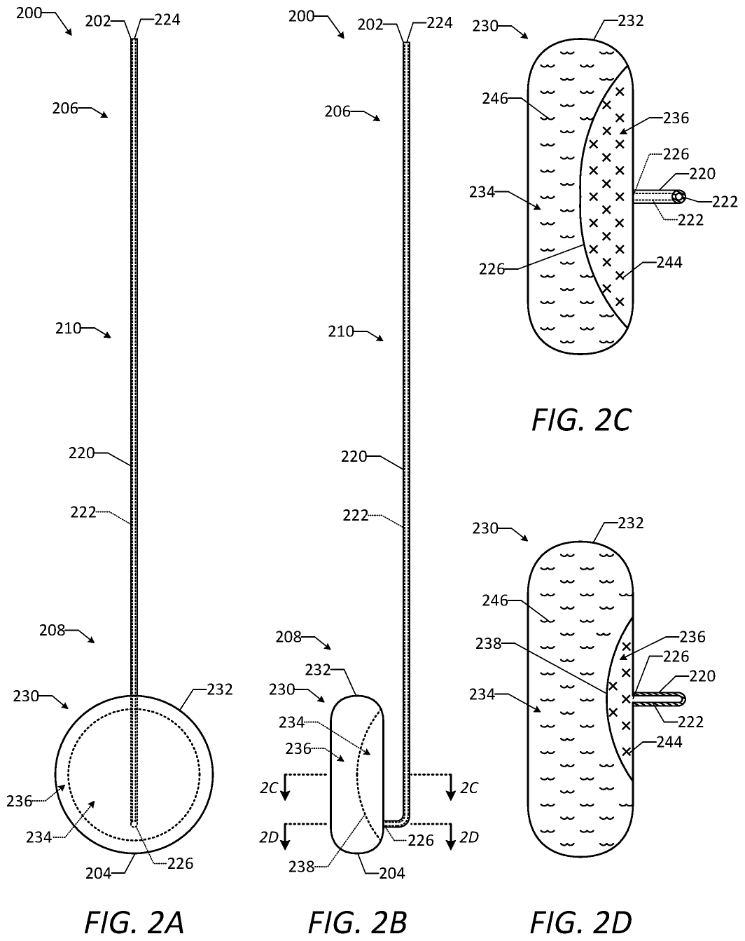

FIGS. 2A-2E illustrate a drug delivery device 200 (which also may be referred to as a “therapeutic agent delivery device” or simply a “device”) in accordance with one or more embodiments of the disclosure. The drug delivery device 200 may be configured to controllably release a drug to a selected region of the lower urinary tract, such as the bladder, of a catheterized patient. During use, a portion of the drug delivery device 200 may be inserted through the patient's urethra and into the bladder to provide a pathway for delivering the drug to the selected region. As described below, the drug delivery device 200 may be used with the urinary catheter 100 to form a urinary catheter and drug delivery system 300 which allows for continuous urine drainage in addition to controlled delivery of a drug to a selected region of the patient's lower urinary tract, such as the bladder, over an extended period of time.

As shown in FIG. 2A, the drug delivery device 200 has an elongated shape including a distal end 202 (which also may be referred to as a “bladder end”) and a proximal end 204 (which also may be referred to as an “external end”). The device 200 includes a distal end portion 206 (which also may be referred to as a “bladder end portion”) extending from the distal end 202 toward the proximal end 204, a proximal end portion 208 (which also may be referred to as an “external end portion”) extending from the proximal end 204 toward the distal end 202, and an intermediate portion 210 (which also may be referred to as a “urethral portion”) extending from the distal end portion 206 to the proximal end portion 208. When the drug delivery device 200 is used to deliver a drug to a selected region of the lower urinary tract, such as the patient's bladder, the distal end portion 206 may be inserted through the urethra and into the bladder, while the intermediate portion 210 resides within the urethra and the proximal end portion 208 resides outside of the patient's body.

The drug delivery device 200 includes a flexible elongate body 220 (which also may be referred to as a “drug delivery body” or a “drug delivery tube”) and a drug reservoir 230 (which also may be referred to as an “external drug reservoir”) attached to the body 220, as shown. The body 220 may extend axially from the distal end 202 toward the proximal end 204 of the device 200 and may be configured to traverse the patient's urethra to reach the bladder. As shown, the body 220 may have an elongated tubular shape and a circular cross-sectional shape, although other shapes of the body 220 may be used. In some embodiments, the body 220 is formed as a capillary tube. The body 220 may include a drug delivery lumen 222 (which also may be referred to as a “primary lumen”) extending axially through the body 220 and configured to allow a drug to pass therethrough from the drug reservoir 230 to the patient's bladder. In particular, the drug delivery lumen 222 may extend from a distal opening 224 (which also may be referred to as a “drug exit opening”) defined in the body 220 to a proximal opening 226 (which also may be referred to as a “drug entry opening”) defined in the body 220. As shown, the distal opening 224 may be defined in or near the distal end of the body 220 and positioned at or near the distal end 202 of the device 200, and the proximal opening 226 may be defined in or near the proximal end of the body 220 and positioned at or near the proximal end 204 of the device 200. In some embodiments, as shown, the drug delivery lumen 222 has a cylindrical shape and a circular axial cross-sectional shape, although other shapes of the drug delivery lumen 222 may be used.

The drug reservoir 230 may include a housing 232 having a plurality of chambers defined therein. In particular, the drug reservoir 230 may include a drug chamber 234 (which also may be referred to as a “therapeutic agent chamber”) and a fluid chamber 236 (which also may be referred to as a “water chamber”) defined therein. The drug chamber 234 may be configured to contain a drug therein, and the fluid chamber 236 may be configured to contain a fluid therein. As shown, the drug chamber 234 and the fluid chamber 236 may be separated by an osmotic barrier 238 (which also may be referred to as a “semi-permeable barrier”). In this manner, the drug chamber 234 may be defined by (i.e., bounded by) a portion of the housing 232 and the osmotic barrier 238, and the fluid chamber 236 may be defined by another portion of the housing 232 and the osmotic barrier 238. As shown, the drug chamber 234 and the fluid chamber 236 may be separated by only the osmotic barrier 238. In other words, a first surface of the osmotic barrier 238 may extend along and define a portion of the drug chamber 234, and an opposite second surface of the osmotic barrier 238 may extend along and define a portion of the fluid chamber 236.

The drug reservoir 230 may include a drug 244 disposed within the drug chamber 234, and a fluid 246 disposed within the fluid chamber 236. In some embodiments, the drug 244 fills or substantially fills the drug chamber 234, and the fluid 246 fills or substantially fills the fluid chamber 236. In some embodiments, the drug 244 is in a solid form. For example, the drug 244 may be in the form of a unitary block that fills or substantially fills the drug chamber 234 or a plurality of tablets, capsules, particles, microparticles, or other solid drug units that fill or substantially fill the drug chamber 234. In other embodiments, the drug 244 is in a semi-solid form or a liquid form that fills or substantially fills the drug chamber 234. In some embodiments, the fluid 246 is sterile water or an aqueous solution (e.g., saline), although other suitable fluids may be used. The term “fluid” as used herein refers to incompressible fluids, i.e., liquids, not gases.

The osmotic barrier 238 may be a semi-permeable wall that is configured to allow the fluid 246 to pass therethrough but to prevent the drug 244 from passing therethrough. For example, the osmotic barrier 238 may be a water-permeable wall. In this manner, the osmotic barrier 238 may allow the fluid 246 to pass therethrough and into the drug chamber 234. In embodiments in which the drug 244 is in a solid or semi-solid form, the fluid 246 may solubilize the drug 244 within the drug chamber 234. The passage of the fluid 246 through the osmotic barrier 238 and into the drug chamber 234 may create osmotic pressure within the drug chamber 234. As shown, the drug delivery lumen 222 of the body 220 may be in fluid communication with the drug chamber 234 via the distal opening 224 of the lumen 222 and a corresponding opening defined in the housing 232 of the drug reservoir 230 adjacent the drug chamber 234. Accordingly, the osmotic pressure created within the drug chamber 234 may drive the drug 244 out of the drug chamber 234, through the drug delivery lumen 222, and out of the drug delivery device 200. In this manner, the drug reservoir 230 may be configured to operate as an osmotic pump to controllably release the drug 244 from the drug delivery device 200 and into a selected region of the lower urinary tract, such as the bladder.

In embodiments, the proximal portion and drug reservoir of the drug delivery device, in use, may be configured to be secured to the patient, particularly for ambulatory patients. For example, the drug reservoir may be strapped to the patient, e.g., about one of the thighs of the patient. For instance, the drug reservoir may be secured within a soft fabric pouch that is connected to a pair of fabric straps connectable to one another by hook-and-loop fasteners or other adjustable fasteners.

Flexible Elongate Body

The flexible elongate body 220 of the drug delivery device 200 is sized and shaped to extend through the urethra of a patient and into the bladder. The body 220 is elastic/flexible such that the body 220 may be easily maneuvered for deployment and positioning within the urethra without undue complications and with minimal discomfort to the patient. When the device 200 is inserted into the patient, the distal end portion 206 is positioned within the bladder, the intermediate portion 210 is positioned within the urethra, and the proximal end portion 208 is positioned outside of the patient's body. In this manner, the drug delivery lumen 222 of the body 220 extends from outside of the patient's body, through the urethra, and into the bladder to facilitate delivery of the drug 244 from outside of the patient's body to the bladder.

The flexible elongate body 220 is generally made of biocompatible polymeric materials known in the art. In some embodiments, the biocompatible polymeric material is silicone or other non-resorbable polymers known in the art. Examples of suitable materials of construction include poly(ethers), poly(acrylates), poly(methacrylates), poly(vinyl pyrolidones), poly(vinyl acetates), poly(urethanes), celluloses, cellulose acetates, poly(siloxanes), poly(ethylene), poly(tetrafluoroethylene) and other fluorinated polymers, poly(siloxanes), copolymers thereof, and combinations thereof. In some embodiments, the body 220 defining the drug delivery lumen 222 is or includes a capillary tube or similar structure. The tube forming the drug delivery lumen 222 may be configured to have suitable wall strength and resistance to compression such that it resists collapse or constriction when deployed in the urethra.

Drug Reservoir

The drug reservoir 220 of the drug delivery device 200 includes the housing 232 and the osmotic barrier 238 which define the chambers of the reservoir 220. As described above, the drug reservoir 220 remains outside of the patient's body during use of the device 200. The drug chamber 234 is defined by (i.e., bounded by) a portion of the housing 232 and the osmotic barrier 238, and the fluid chamber 236 similarly is defined by a portion of the housing 232 and the osmotic barrier 238. The housing 232 includes one or more outer walls that are impermeable to the drug 244 contained within the drug chamber 234 and the fluid 246 contained within the fluid chamber 236. The wall or walls of the housing 232 may be formed of any suitable material, such as a biocompatible polymeric material. In some embodiments, the wall or walls of the housing 232 are formed of the same material as the flexible elongate body 220, although the housing 232 and the body 220 may be formed of different materials in other embodiments. In some embodiments, the wall or walls of the housing 232 are integrally formed with the body 220. For example, the housing 232 and the body 220 may be integrally molded as a unitary structure. In other embodiments, the wall of walls of the housing 232 and the body 220 are separately formed and attached to one another. For example, the housing 232 and the body 220 may be separately formed by extrusion, molding, or a combination thereof, and then attached to other another by a biocompatible adhesive, ultrasonic welding, or other means of attachment.

The osmotic barrier 238 may be a semi-permeable wall, as described above. In particular, the osmotic barrier 238 may be formed of a semi-permeable material that is effective to permit the fluid 246 in the fluid chamber 236 to permeate therethrough and enter the drug chamber 234. The osmotic barrier 238 may be semi-permeable in that, while it is permeable to the fluid 246, such as water, it is substantially or completely impermeable to the drug 244 in the drug chamber 234 and/or an excipient. In this manner, the solubilized drug 244 and excipients cannot diffuse through the osmotic barrier 238 and into the fluid chamber 236. Accordingly, during use of the device 200, the fluid 246 enters the drug chamber 234, solubilizes the drug 244 as well as any excipient (e.g., an osmotic excipient) contained in the drug chamber 234, creating osmotic pressure in the drug chamber 234. The osmotic pressure causes the solubilized drug 244 to be pumped from the drug chamber 234 into and through the drug delivery lumen 222 of the body 220, and directly into the bladder via the distal opening 224. Non-limiting examples of suitable, semi-permeable materials of construction for the osmotic barrier 238 include silicones and polyurethanes known in the art.

Drug

The drug 244 can be any suitable therapeutic, prophylactic, or diagnostic agent. The drug 244 stored in and released from the device 200 may consist only of the pharmaceutically active ingredient (API) or other agent of interest, or the drug 244 may be formulated with one or more pharmaceutically acceptable excipients. The drug 244 may be a biologic. The drug 244 may be a metabolite. As used herein, the term “drug” with reference to any specific drug described herein includes its alternative forms, such as salt forms, free acid forms, free base forms, and hydrates. In some embodiments, the drug is a high solubility drug. As used herein, the term “high solubility” refers to a drug having a solubility above about 10 mg/mL water at 37° C. In other embodiments, the drug is a low solubility drug. As used herein, the term “low solubility” refers to a drug having a solubility from about 0.001 mg/mL to about 10 mg/mL water at 37° C. The solubility of the drug may be affected at least in part by its form and dissolution medium pH. For example, a drug in the form of a water soluble salt may have a high solubility, while the same drug in base form may have a low solubility.

Pharmaceutically acceptable excipients are known in the art and may include lubricants, viscosity modifiers, surface active agents, osmotic agents, diluents, and other non-active ingredients of the formulation intended to facilitate handling, stability, dispersibility, wettability, and/or release kinetics of the drug. The excipient may facilitate loading of solid drug units into the drug reservoir of the device. The excipient also may facilitate forming a therapeutic agent into a solid drug tablet that can be loaded into the drug reservoir. The excipients also may affect the kinetics of drug release from the device, such as by increasing or retarding the solubility or dissolution rate of the drug. In some embodiments, however, the drug release rate is predominately controlled by characteristics of the drug reservoir, such as the thickness and water permeability of the semi-permeable wall.

The drug 244 is to be released from the drug delivery device 200 at a therapeutically effective rate. For some drugs, this may require the addition of one or more excipients, e.g., an osmotic agent to increase water flux, solubilizing or solubility enhancing agent, pH adjusting agent, or stability enhancing agent. Generally, the combination of the solubility of the selected drug in the presence or absence of functional agents, if any, and osmotic pressure flux will determine the release rate and duration, and such combination can be configured for the rate and duration to be within a therapeutically effective range. In embodiments in which the drug is a low solubility drug, the drug may be formulated with an osmotic agent having a higher solubility than the drug, such that the osmotic agent expedites solubilization, causes osmotic pressure flux, and/or subsequent release of the drug. This beneficially allows for the delivery of low solubility or other drugs typically only delivered via diffusion, from osmotic delivery-based devices as described herein.

The drug 244 can be loaded and stored in the device 200 in any suitable form. In some embodiments, the drug 244 is in a solid or semi-solid drug formulation in order to reduce the overall volume of the drug chamber 234 and the overall drug reservoir 230. The semi-solid form may be, for example, an emulsion or suspension; a gel or a paste. The solid form may be, for example, tablets, mini-tablets, pellets, beads, granules, or a powder. In an alternative embodiment, the drug 244 is loaded into the drug chamber 234 in a liquid form. In some embodiments, the drug 244 is preloaded into the drug chamber 234 during manufacture of the drug delivery device 200. In other embodiments, the drug 244 is loaded into the drug chamber 234 by a clinician just prior to use of the device 200.

In some embodiments, the drug 244 includes an antimicrobial agent, such as an antibiotic, antifungal, or antiseptic agent. In this manner, the drug delivery device 200 may be effective in the treatment or prevention of catheter-associated urinary tract infections. In some embodiments, the drug 244 includes an antifibrotic or other agent configured to promote wound healing. In this manner, the drug delivery device 200 may be effective in the prevention of scar tissue formation in a post-surgical setting. In some embodiments, the drug 244 includes an antimuscarinic agent. In this manner, the drug delivery device 200 may be effective in the treatment patients with bladder overactivity (e.g., spinal cord injury patients) who have chronic indwelling catheters. In some embodiments, the drug 244 includes an agent which catalyzes or re-dissolves stones or breaks down biofilms, which may include pharmacological or nonpharmacological agents. In this manner, the drug delivery device 200 may be effective in the prevention of encrustation, stone, or biofilm formation. It will be appreciated that the above-described embodiments of the drug 244 and uses of the drug delivery device 200 are merely examples, as the device 200 may be used to treat or prevent various conditions using various formulations of the drug 244.

In one embodiment, the devices provide pain relief to the patient. A variety of anesthetic agents, analgesic agents, and combinations thereof may be used as the drug 244. In embodiments, the device delivers one or more anesthetic agents. Representative examples of aminoamides or amide-class anesthetics include articaine, bupivacaine, carticaine, cinchocaine, etidocaine, levobupivacaine, lidocaine, mepivacaine, prilocaine, ropivacaine, and trimecaine. Representative examples of aminoesters or ester-class anesthetics include amylocaine, benzocaine, butacaine, chloroprocaine, cocaine, cyclomethycaine, dimethocaine, hexylcaine, larocaine, meprylcaine, metabutoxycaine, orthocaine, piperocaine, procaine, proparacaine, propoxycaine, proxymetacaine, risocaine, and tetracaine. The anesthetic agent may be formulated as a salt, such as a hydrochloride salt, to render them water-soluble, although the anesthetic agent also can be used in free base or hydrate form. Other anesthetics, such as lontocaine, may be used. The drug may be an antimuscarinic compound that exhibits an anesthetic effect, such as oxybutynin or propiverine.

In one embodiment, the analgesic agent includes an opioid. Representative examples of opioid agonists include alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof. Other opioid drugs, such as mu, kappa, delta, and nociception opioid receptor agonists, are contemplated.

Representative examples of other suitable pain relieving agents include such agents as salicyl alcohol, phenazopyridine hydrochloride, acetaminophen, acetylsalicylic acid, flufenisal, ibuprofen, indoprofen, indomethacin, and naproxen.

In one embodiment, the drug delivery device includes a drug 244 which is used to treat inflammatory conditions such as interstitial cystitis (IC), radiation cystitis, painful bladder syndrome, prostatitis, urethritis, post-surgical pain, and kidney stones. Non-limiting examples of drugs for these conditions include lidocaine, glycosaminoglycans (e.g., chondroitin sulfate, sulodexide), pentosan polysulfate sodium (PPS), dimethyl sulfoxide (DMSO), oxybutynin, mitomycin C, heparin, flavoxate, ketorolac, or a combination thereof. For kidney stones, the drug(s) may be selected to treat pain and/or to promote dissolution of renal stones. Other non-limiting examples of drugs that may be used in the treatment of IC include nerve growth factor monoclonal antibody (MAB) antagonists, such as Tanezumab, and calcium channel alpha-2-delta modulators, such as PD-299685 or gabepentin.

In one embodiment, the drug delivery device includes a drug 244 which is used to treat urinary incontinence, frequency, or urgency, including urge incontinence and neurogenic incontinence, as well as trigonitis. Drugs that may be used include anticholinergic agents, antispasmodic agents, anti-muscarinic agents, β-2 agonists, alpha adrenergics, anticonvulsants, norepinephrine uptake inhibitors, serotonin uptake inhibitors, calcium channel blockers, potassium channel openers, and muscle relaxants. Representative examples of drugs for the treatment of incontinence include oxybutynin, S-oxybutytin, emepronium, verapamil, imipramine, flavoxate, atropine, propantheline, tolterodine, rociverine, clenbuterol, darifenacin, terodiline, trospium, hyoscyamin, propiverine, desmopressin, vamicamide, clidinium bromide, dicyclomine HCl, glycopyrrolate aminoalcohol ester, ipratropium bromide, mepenzolate bromide, methscopolamine bromide, scopolamine hydrobromide, iotropium bromide, fesoterodine fumarate, YM-46303 (Yamanouchi Co., Japan), lanperisone (Nippon Kayaku Co., Japan), inaperisone, NS-21 (Nippon Shinyaku Orion, Formenti, Japan/Italy), NC-1800 (Nippon Chemiphar Co., Japan), Z D-6169 (Zeneca Co., United Kingdom), and stilonium iodide.

In one embodiment, the drug delivery device includes a drug 244 which is used to treat urinary tract cancer, such as bladder cancer and prostate cancer. Drugs that may be used include antiproliferative agents, cytotoxic agents, chemotherapeutic agents, or a combination thereof. Representative examples of drugs which may be suitable for the treatment of urinary tract cancer include Bacillus Calmette Guerin (BCG) vaccine, cisplatin, doxorubicin, valrubicin, gemcitabine, mycobacterial cell wall-DNA complex (MCC), methotrexate, vinblastine, thiotepa, mitomycin, fluorouracil, leuprolide, diethylstilbestrol, estramustine, megestrol acetate, cyproterone, flutamide, a selective estrogen receptor modulators (i.e. a SERM, such as tamoxifen), botulinum toxins, and cyclophosphamide. The drug may be a biologic, and it may comprise a monoclonal antibody, a TNF inhibitor, an anti-leukin, or the like. The drug also may be an immunomodulator, such as a TLR agonist, including imiquimod or another TLR7 agonist. The drug also may be a kinase inhibitor, such as a fibroblast growth factor receptor-3 (FGFR3)-selective tyrosine kinase inhibitor, a phosphatidylinositol 3 kinase (PI3K) inhibitor, or a mitogen-activated protein kinase (MAPK) inhibitor, among others or combinations thereof. Other examples include celecoxib, erolotinib, gefitinib, paclitaxel, polyphenon E, valrubicin, neocarzinostatin, apaziquone, Belinostat, Ingenol mebutate, Urocidin (MCC), Proxinium (VB 4845), BC 819 (BioCancell Therapeutics), Keyhole limpet haemocyanin, LOR 2040 (Lorus Therapeutics), urocanic acid, OGX 427 (OncoGenex), and SCH 721015 (Schering-Plough). The drug treatment may be coupled with a conventional radiation or surgical therapy targeted to the cancerous tissue.

In another embodiment, the drug 244 for intravesical cancer treatment may include small molecules, such as Apaziquone, adriamycin, AD-32, doxorubicin, doxetaxel, epirubicin, gemcitabine, HTI-286 (hemiasterlin analogue), idarubicin, γ-linolenic acid, mitozantrone, meglumine, and thiotepa; large molecules, such as Activated macrophages, activated T cells, EGF-dextran, HPC-doxorubicin, IL-12, IFN-α2b, IFN-γ, α-lactalbumin, p53 adenovector, TNFα; combinations, such as Epirubicin+BCG, IFN+farmarubicin, Doxorubicin+5-FU (oral), BCG+IFN, and Pertussis toxin+cystectomy; activated cells, such as macrophages and T cells; intravesical infusions such as IL-2 and Doxorubicin; chemosensitizers, such as BCG-kantifirinolytics (paramethylbenzoic acid or aminocaproic acid) and Doxorubicin+verapimil; diagnostic/imaging agents, such as Hexylaminolevulinate, 5-aminolevulinic acid, Iododexyuridine, HMFG1 Mab+Tc99m; and agents for the management of local toxicity, such as Formaline (hemorrhagic cystitis).

In one embodiment, the drug delivery device includes a drug 244 which is used to treat infections involving the bladder, the prostate, and the urethra. Antibiotics, antibacterial, antifungal, antiprotozoal, antiseptic, antiviral and other antiinfective agents can be administered for treatment of such infections. Representative examples of drugs for the treatment of infections include mitomycin, ciprofloxacin, norfloxacin, ofloxacin, methanamine, nitrofurantoin, ampicillin, amoxicillin, nafcillin, trimethoprim, sulfonamides trimethoprimsulfamethoxazole, erythromycin, doxycycline, metronidazole, tetracycline, kanamycin, penicillins, cephalosporins, and aminoglycosides.

In one embodiment, the drug delivery device includes a drug 244 which is used to treat fibrosis of a genitourinary site, such as the bladder or uterus. Representative examples of drugs for the treatment of fibroids include pentoxphylline (xanthine analogue), antiTNF, antiTGF agents, GnRH analogues, exogenous progestins, antiprogestins, selective estrogen receptor modulators, danazol and NSAIDs.

In one embodiment, the drug delivery device includes a drug 244 which is used to treat neurogenic bladder. Representative examples of drugs for the treatment of neurogenic bladder include analgesics or anaesthetics, such as lidocaine, bupivacaine, mepivacaine, prilocaine, articaine, and ropivacaine; anticholinergics; antimuscarinics such as oxybutynin or propiverine; a vanilloid, such as capsaicin or resiniferatoxin; antimuscarinics such as ones that act on the M3 muscarinic acetylcholine receptor (mAChRs); antispasmodics including GABAB agonists such as baclofen; botulinum toxins; capsaicins; alpha-adrenergic antagonists; anticonvulsants; serotonin reuptake inhibitors such as amitriptyline; and nerve growth factor antagonists. In various embodiments, the drug may be one that acts on bladder afferents or one that acts on the efferent cholinergic transmission, as described in Reitz et al., Spinal Cord 42:267-72 (2004).

In one embodiment, the drug 244 is selected from those known for the treatment of incontinence due to neurologic detrusor overactivity and/or low compliant detrusor. Examples of these types of drugs include bladder relaxant drugs (e.g., oxybutynin (antimuscarinic agent with a pronounced muscle relaxant activity and local anesthetic activity), propiverine, impratroprium, tiotropium, trospium, terodiline, tolterodine, propantheline, oxyphencyclimine, flavoxate, and tricyclic antidepressants; drugs for blocking nerves innervating the bladder and urethra (e.g., vanilloids (capsaicin, resiniferatoxin), botulinum-A toxin); or drugs that modulate detrusor contraction strength, micturition reflex, detrusor sphincter dyssynergia (e.g., GABAb agonists (baclofen), benzodiazapines). The drug may be selected from those known for the treatment of incontinence due to neurologic sphincter deficiency. Examples of these drugs include alpha adrenergic agonists, estrogens, beta-adrenergic agonists, tricyclic antidepressants (imipramine, amitriptyline). The drug may be selected from those known for facilitating bladder emptying (e.g., alpha adrenergic antagonists (phentolamine) or cholinergics). The drug may be selected from among anticholinergic drugs (e.g., dicyclomine), calcium channel blockers (e.g., verapamil) tropane alkaloids (e.g., atropine, scopolamine), nociceptin/orphanin FQ, and bethanechol (e.g., m3 muscarinc agonist, choline ester).

Urinary Catheter and Drug Delivery System

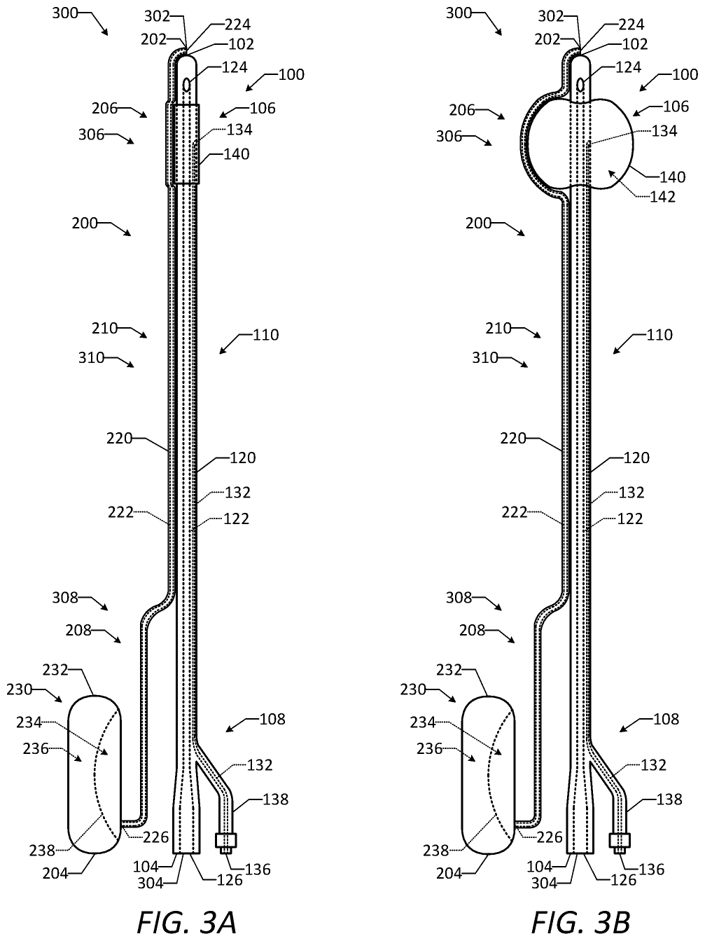

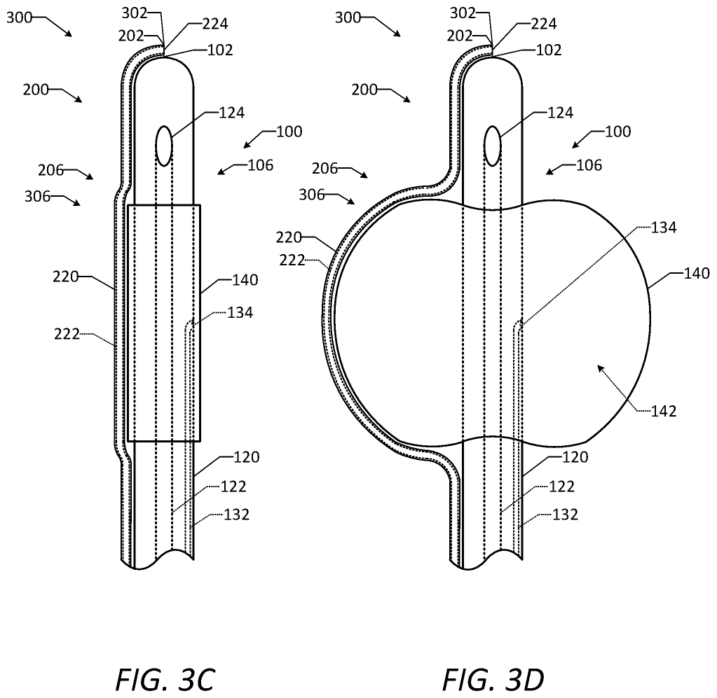

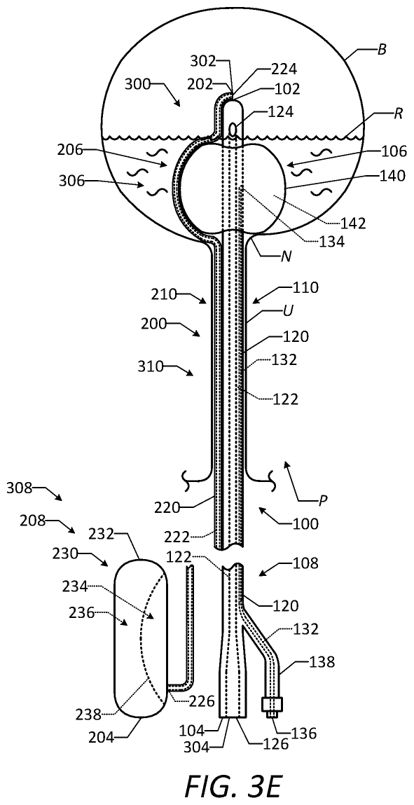

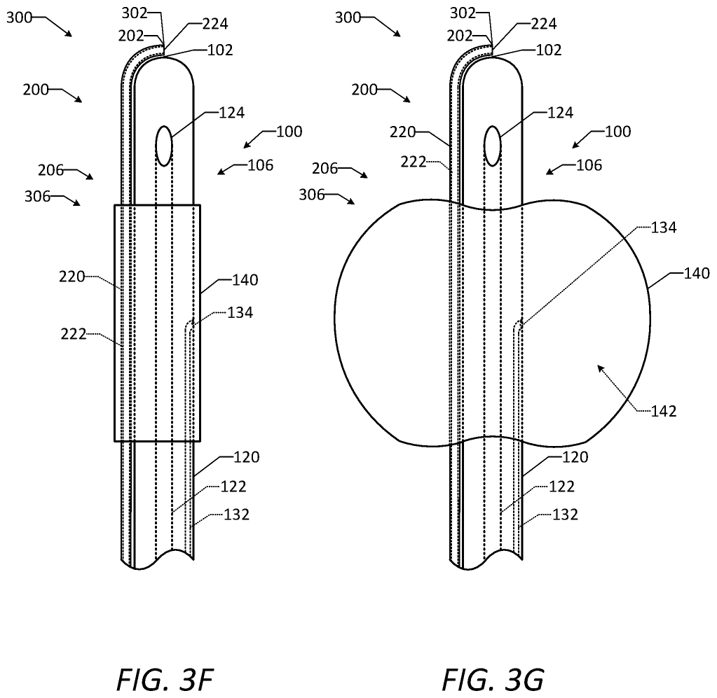

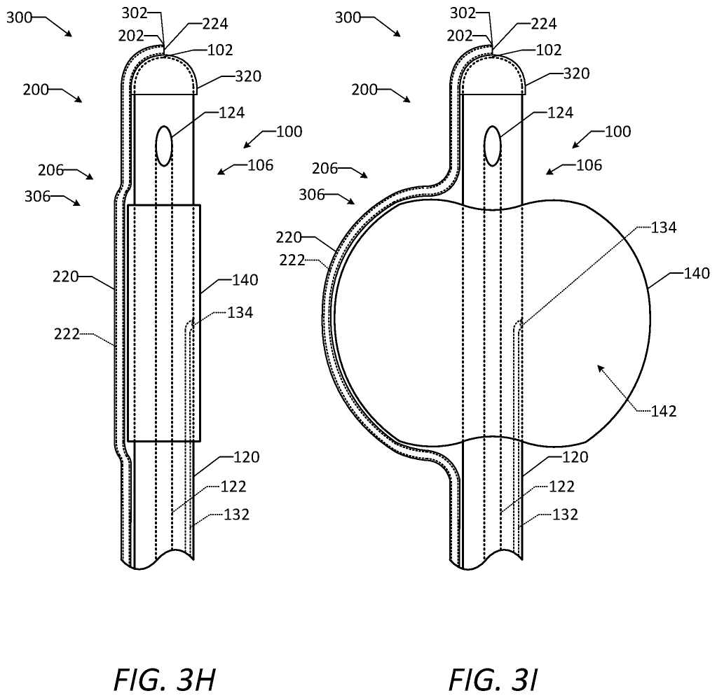

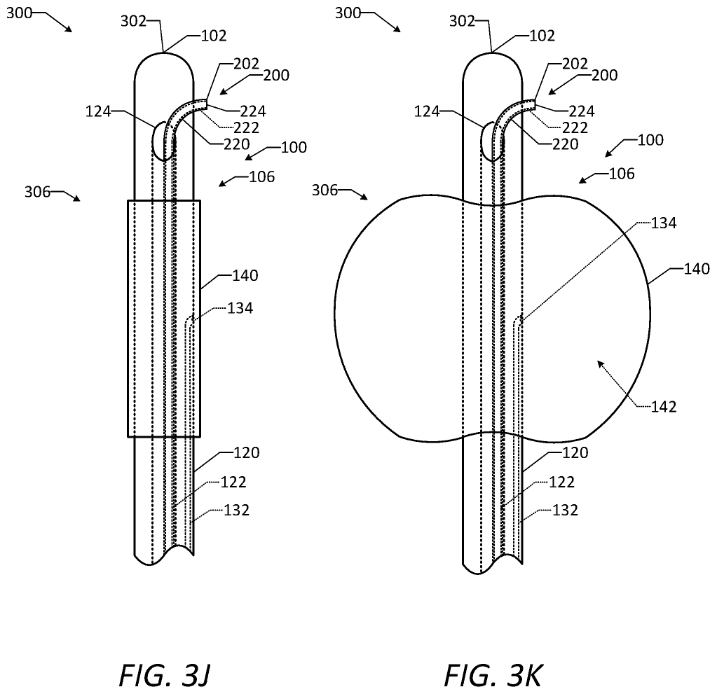

FIGS. 3A-3I illustrate a urinary catheter and drug delivery system 300 (which also may be referred to as simply a “system”) in accordance with one or more embodiments of the disclosure. As shown, the system 300 includes the urinary catheter 100 and the drug delivery device 200, which each may provide the functions described above. The drug delivery device 200 may be permanently or removably attached to the urinary catheter 100. In this manner, the urinary catheter 100 may serve as a support structure for positioning and supporting the drug delivery device 200 relative to the patient for drug delivery. Ultimately, the urinary catheter and drug delivery system 300 may allow for continuous urine drainage in addition to controlled delivery of a drug to a selected region of the patient's lower urinary tract, such as the bladder, over an extended period of time.

As shown in FIG. 3A, the urinary catheter and drug delivery system 300 has an elongated shape including a distal end 302 (which also may be referred to as a “bladder end”) and a proximal end 304 (which also may be referred to as an “external end”). The system 300 includes a distal end portion 306 (which also may be referred to as a “bladder end portion”) extending from the distal end 302 toward the proximal end 304, a proximal end portion 308 (which also may be referred to as an “external end portion”) extending from the proximal end 304 toward the distal end 302, and an intermediate portion 310 (which also may be referred to as a “urethral portion”) extending from the distal end portion 306 to the proximal end portion 308. As shown, the distal end portion 306 includes the distal end portion 106 of the urinary catheter 100 and the distal end portion 206 of the drug delivery device 200, the proximal end portion 308 includes the proximal end portion 108 of the catheter 100 and the proximal end portion 208 of the device 200, and the intermediate portion 310 includes the intermediate portion 110 of the catheter 100 and the intermediate portion 210 of the device 200. When the urinary catheter and drug delivery system 300 is used for urine drainage from a patient's bladder and drug delivery to the bladder, the distal end portion 306 may be inserted through the urethra and into the bladder, while the intermediate portion 310 resides within the urethra and the proximal end portion 308 resides outside of the patient's body.

In some embodiments, the drug delivery device 200 is permanently attached to the urinary catheter 100 such that the system 300 is a permanent assembly. As shown in FIGS. 3A-3D, the device 200 may extend along at least a portion of the length of the catheter 100 and be attached thereto. In particular, the device body 220 may extend along at least a portion of the catheter 100 and be attached thereto. In some embodiments, as shown, the device body 220 extends along the external surface of the catheter body 120 and is attached thereto. For example, the device body 220 may be attached to the external surface of one or more, or all, of the distal end portion of the catheter body 120 (i.e., distally with respect to the balloon 140), the intermediate portion of the catheter body 120 (i.e., proximally with respect to the balloon 140), and the proximal end portion of the catheter body 120. In some embodiments, the device body 220 extends along the external surface of the balloon 140 and is attached thereto, either in addition to or instead of being attached to one or more portions of the catheter body 120. In some embodiments, as shown, at least part of the proximal end portion of the device body 220 is separate from (i.e., not attached to) a respective part of the proximal end portion of the catheter body 120. In this manner, such parts of the catheter body 120 and the device body 220, as well as the drug reservoir 230, may be separately manipulated during use of the system 300. The attached portions of the drug delivery device 200 and the urinary catheter 100 may be permanently attached to one another by a biocompatible adhesive, ultrasonic welding, or other suitable means of attachment.

In other embodiments, the drug delivery device 200 is removably attached to the urinary catheter 100 such that the system 300 is a separable assembly. In this manner, the device 200 may be attached to the catheter 100 when drug delivery is desired and removed from the catheter 100 when drug delivery is not needed. In such embodiments, the drug delivery device 200 may include one or more releasable fasteners, such as caps, clips, bands, straps, or other types of mechanical fasteners configured for releasably attaching the device 200 to the catheter 100. Alternatively, the catheter 100 may include one or more releasable fasteners, such as caps, clips, bands, straps, or other types of mechanical fasteners configured for releasably attaching the device 200 to the catheter 100. According to various embodiments, the releasable fasteners may attach the device body 220 to the catheter body 120 and/or the balloon 140 along one or more, or all, of the distal end portion 306, the proximal end portion 308, and the intermediate portion 310 of the system 300.

When the drug delivery device 200 is attached, either permanently or removably, to the urinary catheter 100, the distal opening 224 of the drug delivery lumen 222 may be positioned along the distal end portion 306 of the system 300. In some embodiments, as shown in FIGS. 3A-3D, the distal opening 224 is positioned at the distal end 302 of the system 300. In other embodiments, the distal opening 224 is positioned near but proximally spaced apart from the distal end 302 of the system 300. In some embodiments, the distal opening 224 of the drug delivery lumen 222 is positioned adjacent the distal opening 124 of the drainage lumen 122. In other embodiments, the distal opening 224 of the drug delivery lumen 222 is distally or proximally spaced apart from the distal opening 124 of the drainage lumen 122. In some embodiments, as shown, the distal opening 224 of the drug delivery lumen 222 faces a first direction, and the distal opening 124 of the drainage lumen 122 faces a second direction that is different from the first direction. For example, the first direction may be transverse to the second direction or may be opposite the second direction. In other embodiments, the distal opening 224 of the drug delivery lumen 222 and the distal opening 124 of the drainage lumen 122 face the same direction. In some embodiments, as shown, the distal opening 224 of the drug delivery lumen 222 is distally spaced apart from the balloon 140. In other embodiments, the distal opening 224 of the drug delivery lumen 222 is positioned along the external surface of the balloon 140.

FIG. 3E illustrates use of the urinary catheter and drug delivery system 300 to allow urine to drain from the bladder B of a patient P and also deliver a drug into the bladder B. With the drug delivery device 200 attached, either permanently or removably, to the urinary catheter 100 and the balloon 140 in the collapsed configuration, the distal end portion 306 of the system 300 may be inserted through the urethra U and into the bladder B. In particular, distal end portion 306 of the system 300 may be inserted such that the balloon 140 is disposed within the bladder B adjacent the bladder neck N, while the intermediate portion 310 of the system 300 is disposed within the urethra U and the proximal end portion 308 of the system 300 is disposed outside of the body of the patient P. Fluid, such as sterile water, then may be passed through the inflation lumen 132 and into the cavity 142 to inflate the balloon 140 to the expanded configuration, such that the balloon 140 forms a seal against the bladder neck N.

With the catheter 100 positioned as shown in FIG. 3E, urine may freely enter the distal opening 124 of the drainage lumen 122, pass through the drainage lumen 122, and be collected in a collection bag attached to the proximal end 104 of the catheter 100. As shown, some residual urine R may remain in the bladder B due to the position of the distal opening 124 of the drainage lumen 122 relative to the bladder neck N With the drug delivery device 200 positioned as shown in FIG. 3E, the fluid 246 within the fluid chamber 236 may permeate through the osmotic barrier 238 and into the drug chamber 234. In this manner, the fluid 246 may solubilize the drug 244 within the drug chamber 234 and create osmotic pressure within the drug chamber 234. The osmotic pressure created may drive the drug 244 out of the drug chamber 234, through the drug delivery lumen 222, and out of the drug delivery device 200 into the bladder B. In particular, the drug 244 may be released from the drug delivery lumen 222, via the distal opening 224, directly into the bladder B. In other words, the distal opening 224 may be in direct fluid communication with the bladder B, such the drug 244 passes directly from the drug delivery lumen 222 into the bladder B. In this manner, in reaching the bladder B, the drug 244 does not pass through any additional components or features positioned between the distal opening 224 and the bladder B. As described above, the drug reservoir 230 may operate as an osmotic pump to controllably release the drug 244 from the drug delivery device 200 and into the bladder over an extended period of time, such as multiple days, weeks, or months, depending on the drug payload of the reservoir 230. Because the drug reservoir 230 is disposed outside of the patient's body, the drug chamber 234 may be sufficiently large to accommodate the drug payload necessary for controlled drug delivery over such an extended period of time.

FIGS. 3F and 3G illustrate another version of the urinary catheter and drug delivery system 300, which includes the drug delivery device 200 and the urinary catheter 100. As shown, the device body 220 may extend between the balloon 140 and the external surface of the catheter body 120, instead of running along the outside of the balloon 140 as illustrated in FIGS. 3C and 3D. In this manner, the balloon 140 may help secure the distal end portion 206 of the drug delivery device 200 to the urinary catheter 100. In some embodiments, the balloon 140 includes a passageway defined therein and configured to allow a portion of the device body 220 to be positioned therein. In other embodiments, the body 120 of the urinary catheter 100 includes a passageway defined therein and configured to allow a portion of the device body 220 to be positioned therein. In still other embodiments, a passageway may be defined between the balloon 140 and the body 120 of the urinary catheter 100 and configured to allow a portion of the device body 220 to be positioned therein. In some embodiments, the drug delivery device 200 is removably attached to the urinary catheter 100 to provide the arrangement shown in FIGS. 3F and 3G. For example, the device body 220 may be slid through the passageway defined by the balloon 140 and/or the body 120 and secured therewithin, e.g., by frictional engagement. In other embodiments, the drug delivery device 200 is permanently attached to the urinary catheter 100 to provide the illustrated arrangement. For example, the device body 220 may be positioned through the passageway defined by the balloon 140 and/or the body 120 and permanently secured to the balloon 140 and/or the body 120 by one or more suitable means of attachment.

FIGS. 3H and 3I illustrate yet another version of the urinary catheter and drug delivery system 300, which includes the drug delivery device 200 attached to the urinary catheter 100 via a cap 320. In some embodiments, the cap 320 is a part of the drug delivery device 200 and is configured for removable attachment to the urinary catheter 100. For example, the cap 320 may be permanently attached to the distal end portion of the device body 220 and configured for removable attachment to the distal end portion of the catheter body 120, as shown. In this manner, the drug delivery device 200 may be removably attached to the catheter 100, via the cap 320, when desired for drug delivery. In such embodiments, the cap 320 may be press-fitted onto the distal end of the catheter body 120, adhered to the distal end of the catheter body 120 via a releasable biocompatible adhesive, or otherwise removably attached to the distal end of the catheter body 120. In other embodiments, the cap 320 is permanently attached to both the device body 220 and the catheter body 120.

FIGS. 3J and 3K illustrate still another version of the urinary catheter and drug delivery system 300, which includes the drug delivery device 200 and the urinary catheter 100. As shown, the device body 220 may extend through the drainage lumen 122 of the catheter body 120, such that the distal opening 224 of the drug delivery lumen 222 is positioned outside of the drainage lumen 122. In some embodiments, the drug delivery device 200 is removably attached to the urinary catheter 100 to provide the arrangement shown in FIGS. 3J and 3K. In other embodiments, the drug delivery device 200 is permanently attached to the urinary catheter 100 to provide the illustrated arrangement. For example, the device body 220 may be permanently attached to the wall of the drainage lumen 122. In still other embodiments, the drug delivery device 200 is not attached to the urinary catheter 100 at all, as the device body 220 is merely inserted through the drainage lumen 122. In this manner, the relative position of the distal opening 224 of the drug delivery lumen 222 with respect to the distal opening 124 of the drainage lumen 122 may be adjusted, as desired.

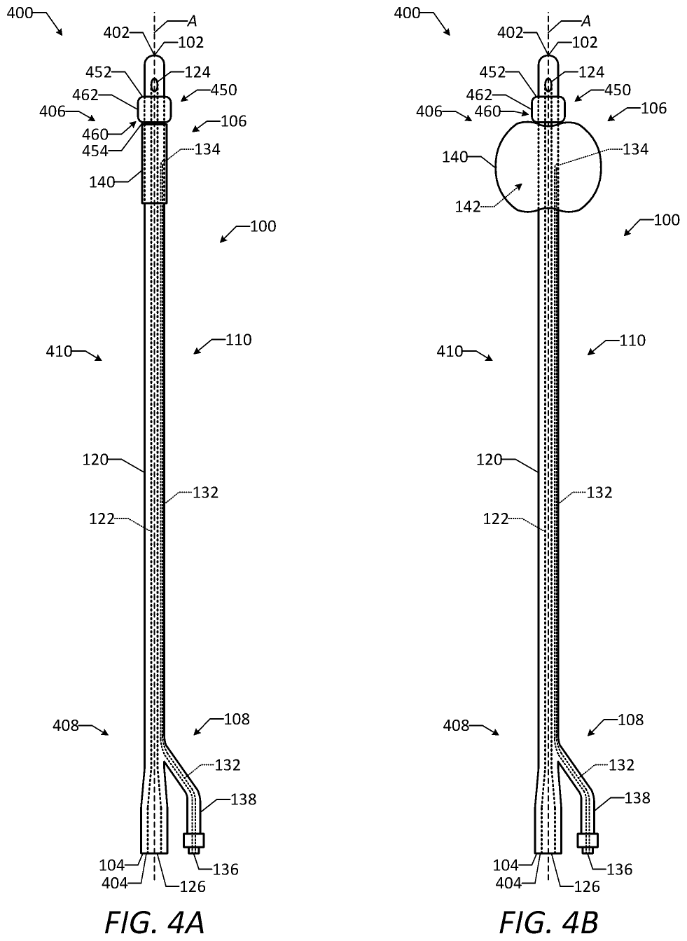

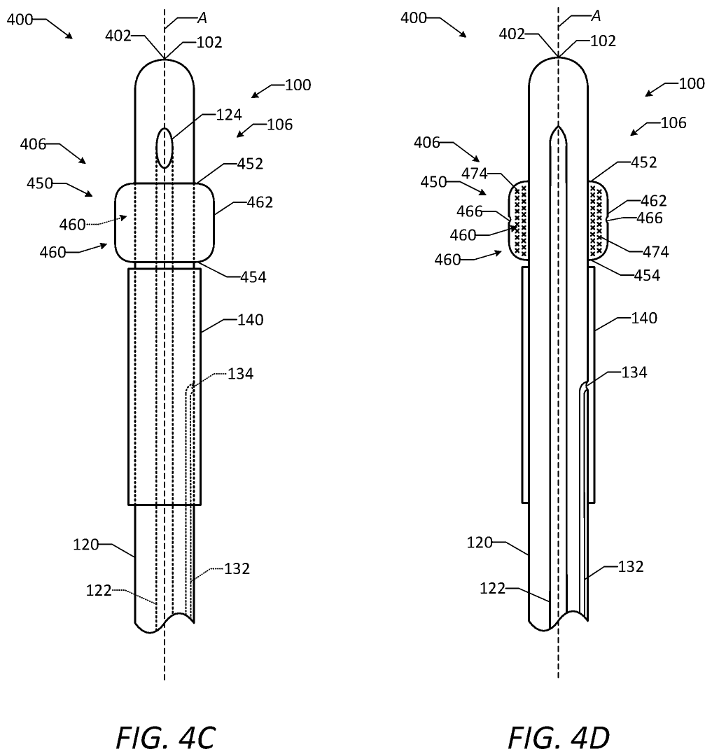

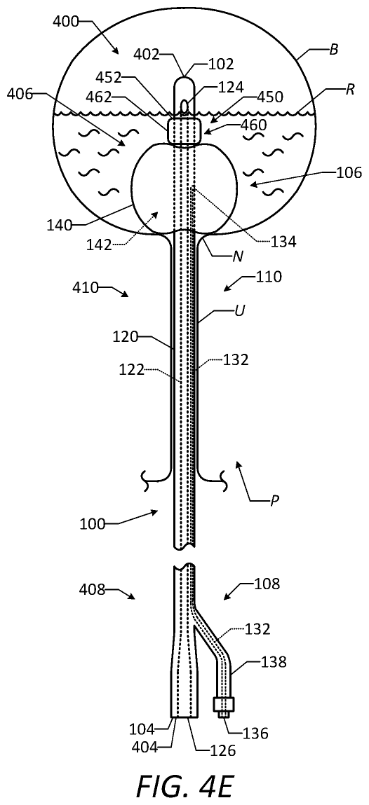

FIGS. 4A-4E illustrate a urinary catheter and drug delivery system 400 (which also may be referred to as simply a “system”) in accordance with one or more embodiments of the disclosure. As shown, the system 400 includes the urinary catheter 100, which may provide the functions described above, and a drug delivery device 450 (which also may be referred to as a “therapeutic agent delivery device” or simply a “device”) attached to the urinary catheter 100. The drug delivery device 450 may be permanently or removably attached to the urinary catheter 100. In this manner, the urinary catheter 100 may serve as a support structure for positioning and supporting the drug delivery device 450 relative to the patient for drug delivery. Ultimately, the urinary catheter and drug delivery system 400 may allow for continuous urine drainage in addition to controlled delivery of a drug to a selected region of the patient's lower urinary tract, such as the bladder, over an extended period of time.

As shown in FIG. 4A, the urinary catheter and drug delivery system 400 has an elongated shape including a distal end 402 (which also may be referred to as a “bladder end”) and a proximal end 404 (which also may be referred to as an “external end”) positioned along a longitudinal axis A of the system 400. The system 400 includes a distal end portion 406 (which also may be referred to as a “bladder end portion”) extending from the distal end 402 toward the proximal end 404 along the longitudinal axis A, a proximal end portion 408 (which also may be referred to as an “external end portion”) extending from the proximal end 404 toward the distal end 402 along the longitudinal axis A, and an intermediate portion 410 (which also may be referred to as a “urethral portion”) extending axially from the distal end portion 406 to the proximal end portion 408. As shown, the distal end portion 406 includes the distal end portion 106 of the urinary catheter 100 and the drug delivery device 450, the proximal end portion 408 includes the proximal end portion 108 of the catheter 100, and the intermediate portion 410 includes the intermediate portion 110 of the catheter 100. When the urinary catheter and drug delivery system 400 is used for urine drainage from the a patient's bladder and drug delivery to the bladder, the distal end portion 406 may be inserted through the urethra and into the bladder, while the intermediate portion 410 resides within the urethra and the proximal end portion 408 resides outside of the patient's body.

The drug delivery device 450 may be configured to controllably release a drug to a selected region of the lower urinary tract, such as the bladder, of a catheterized patient. During use, the entire drug delivery device 450 may be inserted through the patient's urethra and into the bladder to provide a mechanism for delivering the drug to the selected region. As shown in FIG. 4A, the drug delivery device 450 has an annular shape extending around and coaxial with the longitudinal axis A of the system 400. In this manner, the drug delivery device 450 may extend entirely around a distal end portion of the body 120 of the catheter 100. Although the drug delivery device 450 is illustrated as having an annular or toroidal shape, it will be appreciated that other shapes of the device 450 may be used in other embodiments. As shown, the drug delivery device 450 includes a distal end 452 and a proximal end 454 opposite the distal end 452. When the drug delivery device 450 is used to deliver a drug to a selected region of the lower urinary tract, such as the patient's bladder, the entire device 450 may be inserted through the urethra, such that the distal end 452 and the proximal end 454 both reside in the bladder.

As shown, the drug delivery device 450 may include, or may be formed as, a drug reservoir 460 attached to the distal end portion of the catheter body 120. In some embodiments, the drug reservoir 460 is permanently attached to the catheter body 120. In other embodiments, the drug reservoir 460 is removably attached to the catheter body 120, for example, by one or more releasable fasteners. The drug reservoir 460 may have an annular or toroidal shape, although other shapes of the drug reservoir 460 may be used.

As shown, the drug reservoir 460 may be positioned axially between the distal opening 124 of the drainage lumen 122 and the balloon 140. In other words, the distal end of the drug reservoir 460 may be positioned proximally with respect to the distal opening 124, and the proximal end of the drug reservoir 460 may be positioned distally with respect to the balloon 140. In some embodiments, as shown, the distal end of the drug reservoir 460 is axially spaced apart from the distal opening 124, and the proximal end of the drug reservoir 460 is axially spaced apart from the balloon 140. Alternatively, the distal end of the drug reservoir 460 may abut the distal opening 124, and/or the proximal end of the drug reservoir 460 may abut the balloon 140. By locating the drug delivery device below the distal opening 124 and above the balloon 140, the drug delivery device advantageously will be positioned in, or in contact with, the residual volume of urine in the bladder, which tends to remain below the drainage opening, as the drug delivery device 450 relies on the urine the medium for transfer of the drug from the device to the tissues of the patient's bladder.

The drug reservoir 460 may include a housing 462 having one or more chambers defined therein. In particular, the drug reservoir 460 may include a drug chamber 464 (which also may be referred to as a “therapeutic agent chamber”) defined therein. Although the illustrated embodiment includes only a single drug chamber 464, the drug reservoir 460 may include two or more drug chambers 464 in other embodiments. The drug chamber 464 may be configured to contain a drug therein. In some embodiments, as shown, the drug chamber 464 is defined by (i.e., bounded by) a portion of the housing 462 and a portion of the catheter body 120. In particular, the drug chamber 464 may be defined by an internal surface of the outer circumferential wall of the housing 462 and an external surface of the sidewall of the catheter body 120, as shown. In other embodiments, the drug chamber 464 is defined entirely by the housing 462. For example, the housing 462 may include an inner circumferential wall extending along and around the external surface of the sidewall of the catheter body 120, such that the drug chamber 464 is defined by and between the internal surface of the outer circumferential wall and the external surface of the inner circumferential wall of the housing 462.

As shown in FIG. 4D, the drug reservoir 460 may include a drug 474 disposed within the drug chamber 464. In some embodiments, the drug 474 fills or substantially fills the drug chamber 464. In some embodiments, the drug 474 is in a solid form. For example, the drug 474 may be in the form of a unitary block that fills or substantially fills the drug chamber 464 or a plurality of tablets, mini-tablets, pellets, beads, granules, a powder, or other solid drug units that fill or substantially fill the drug chamber 464. In other embodiments, the drug 474 is in a semi-solid form or a liquid form that fills or substantially fills the drug chamber 464. The semi-solid form may be, for example, an emulsion or suspension; a gel or a paste. In some embodiments, the drug 474 is preloaded into the drug chamber 464 during manufacture of the drug delivery device 450. In other embodiments, the drug 474 is loaded into the drug chamber 464 by a clinician just prior to use of the drug delivery device 450. The drug 474 may be any suitable therapeutic, prophylactic, or diagnostic agent. According to various embodiments, the drug 474 may be or may include any of the agents described above with respect to the drug 244, although still other agents may be used in other embodiments.

The wall or walls of the housing 462 may be formed of any suitable material, such as a biocompatible polymeric material. In some embodiments, the wall or walls of the housing 462 are formed of the same material as the catheter body 120, although the housing 462 and the catheter body 120 may be formed of different materials in other embodiments. In some embodiments, the wall or walls of the housing 462 are integrally formed with the catheter body 120. For example, the housing 462 and the catheter body 120 may be integrally molded as a unitary structure. In other embodiments, the wall of walls of the housing 462 and the catheter body 120 are separately formed and attached to one another. For example, the housing 462 and the catheter body 120 may be separately formed by extrusion, molding, or a combination thereof, and then attached to other another by a biocompatible adhesive, ultrasonic welding, or other means of attachment. In some embodiments, the housing 462 is formed of an elastomeric or flexible material to permit some deformation of the housing 462, which may ease insertion of the drug delivery device 450 through the patient's urethra and into the bladder. The material used to form the housing 462 also may be water permeable or porous so that solubilizing fluid (e.g., urine) can enter the drug chamber 464 to solubilize the drug 474 once the drug delivery device 450 is positioned in the bladder. For example, silicone or another biocompatible elastomeric material may be used. In various embodiments, depending at least in part on the selected mechanism of drug release for the selected drug, the housing wall(s) may be formed of a thermoplastic elastomeric material, such as one or more suitable thermoplastic polyurethanes known in the art. Examples of such materials include Tecophilic™, HydroThane™, Hydromed™, Dryflex™, Carbothane™, Tecoflex™, Isoplast™, Pellethane™, Tecoplast™, Tecothane™, or a combination thereof.

The housing 462 is configured to allow the drug 474 to be released from the drug chamber 464 and into the patient's bladder. The drug release mechanism may be osmosis or diffusion through orifice(s) or permeation through the reservoir membrane with or without an orifice. The release rate of the drug 474 from the drug chamber 464 generally is controlled by the design of the combination of the device components, including but not limited to the materials, dimensions, surface area, and apertures of the housing 462, as well as the particular drug formulation and total mass of drug load, among others.

In some embodiments, the housing 462 includes one or more apertures 466 extending through the wall or walls of the housing 462 and in fluid communication with the drug chamber 464. In some embodiments in which an aperture 466 is provided, the aperture 466 may be temporarily closed by a degradable or dissolvable timing membrane, which may control the initiation of release of the drug 474 from the drug chamber 464.

In some embodiments, the drug reservoir 460 operates as an osmotic pump. In such embodiments, the housing 462 may be formed from a water permeable material, such as a silicone, which may act as a semi-permeable membrane, permeable to water but not to the selected drug in solubilized form. Following positioning of the drug delivery device 460 within the bladder, urine diffuses through a wall of the housing 462, enters the drug chamber 464, and solubilizes the drug 474. Solubilized drug 474 then is dispensed at a controlled rate out of the drug chamber 464 through the one or more apertures 466, driven by osmotic pressure in the drug chamber 464. The delivery rate and overall performance of the osmotic pump is affected by device parameters, such as the surface area of the housing 462; the permeability to liquid of the material used to form the housing 462; the size and placement of the apertures 466; and the drug formulation dissolution profile, among other factors.

In other embodiments, the drug delivery device 450 may operate essentially by diffusion of the drug 474 from the housing 462 through (i) one or more discrete apertures 466 formed in the wall or walls of the housing 462, (ii) through the wall or walls of the housing 462 itself, which may be permeable to the drug 474, or (iii) a combination thereof. In embodiments in which diffusion occurs through the wall or walls of the housing 462, the apertures 466 or passing pores may not be included. In still other embodiments, the drug delivery device 450 may operate by a combination of osmosis and diffusion.

In some embodiments, the housing 462 is non-resorbable. For example, the housing 462 may be formed of a medical grade silicone. In another example, the housing may be formed of a thermoplastic elastomer, as described above. Other examples of suitable non-resorbable materials include synthetic polymers selected from poly(ethers), poly(acrylates), poly(methacrylates), poly(vinyl pyrolidones), poly(vinyl acetates), poly(urethanes), celluloses, cellulose acetates, poly(siloxanes), poly(ethylene), poly(tetrafluoroethylene) and other fluorinated polymers, poly(siloxanes), copolymers thereof, and combinations thereof.

In some embodiments, the housing 462 is bioerodible. In one embodiment of a bioerodible housing 462, the housing 462 is formed of a biodegradable or bioresorbable polymer. Examples of suitable such materials include synthetic polymers selected from poly(amides), poly(esters), poly(ester amides), poly(anhydrides), poly(orthoesters), polyphosphazenes, pseudo poly(amino acids), poly(glycerol-sebacate)(PGS), copolymers thereof, and mixtures thereof.

The size, number, and placement of the apertures 466 may be selected to provide a controlled rate of release of the drug 474. A drug delivery device 450 that operates primarily as an osmotic pump may have one or more apertures 466 sized small enough to reduce diffusion of the drug 474 through the aperture(s) 466, yet large enough and spaced appropriately along the housing 462 to manage the buildup of hydrostatic pressure in the housing 462. Within these constraints, the size and number of apertures 466 for a single drug delivery device 450 can be varied to achieve a selected release rate. In an exemplary embodiment, the device includes a single aperture having a diameter between about 20 μm and about 500 μm. In embodiments where the drug delivery device 450 operates primarily by diffusion, the apertures 466, if present, may be in this range or larger.

In some embodiments, the housing 462 may not have any apertures, in which case the drug 474 may be released via a release mechanism other than osmosis, such as diffusion through the wall or walls of the housing 462. Similarly, a drug delivery device 450 having multiple discrete drug chambers 464 may have apertures 466 associated with all, some, or none of the drug chambers 464, in which cases release from the different drug chambers 464 may occur via different release mechanisms.

FIG. 4E illustrates use of the urinary catheter and drug delivery system 400 to allow urine to drain from the bladder B of a patient P and also deliver a drug into the bladder B. With the drug delivery device 450 attached, either permanently or removably, to the urinary catheter 100 and the balloon 140 in the collapsed configuration, the distal end portion 406 of the system 400 may be inserted through the urethra U and into the bladder B. In particular, distal end portion 406 of the system 400 may be inserted such that the balloon 140 is disposed within the bladder B adjacent the bladder neck N, while the intermediate portion 410 of the system 400 is disposed within the urethra U and the proximal end portion 408 of the system 400 is disposed outside of the body of the patient P. Fluid, such as sterile water, then may be passed through the inflation lumen 132 and into the cavity 142 to inflate the balloon 140 to the expanded configuration, such that the balloon 140 forms a seal against the bladder neck N.

With the catheter 100 positioned as shown in FIG. 4E, urine may freely enter the distal opening 124 of the drainage lumen 122, pass through the drainage lumen 122, and be collected in a collection bag attached to the proximal end 104 of the catheter 100. The urine flows by gravity. As shown, some residual urine R may remain in the bladder B due to the position of the distal opening 124 of the drainage lumen 122 relative to the bladder neck N With the drug delivery device 450 positioned between the distal opening 124 of the drainage lumen 122 and the balloon 140, as shown in FIG. 4E, the device 450 may reside within the residual urine R. In embodiments in which the housing 462 is formed of a water permeable material, some of the residual urine R may permeate through the wall or walls of the housing 462 and solubilize the drug 474 within the drug chamber 464. Ultimately, the drug delivery device 450 may controllably release the drug 474 into the bladder B via one or more apertures 466 or pores or through the wall or walls of the housing 462, according to one or more of the release mechanisms described above. In particular, the drug delivery device 450 may release the drug 474 directly into the bladder B. The drug 474 may be released from the drug delivery device 450 and into the bladder B over an extended period of time, such as multiple days, weeks, or months, depending on the drug payload of the drug reservoir 460. If the drug 474 present in the drug chamber 464 becomes depleted, the drug chamber 464 may be refilled, a new drug delivery device 450 may be attached to the urinary catheter 100 upon removal of the urinary catheter and drug delivery system 400, or a new system 400 may be used for further drug delivery.

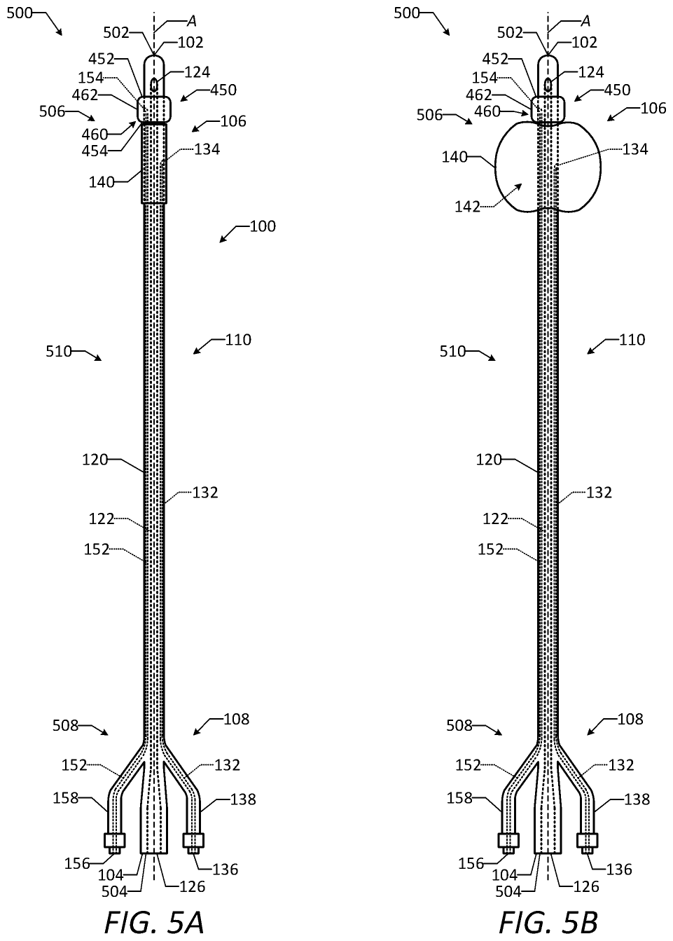

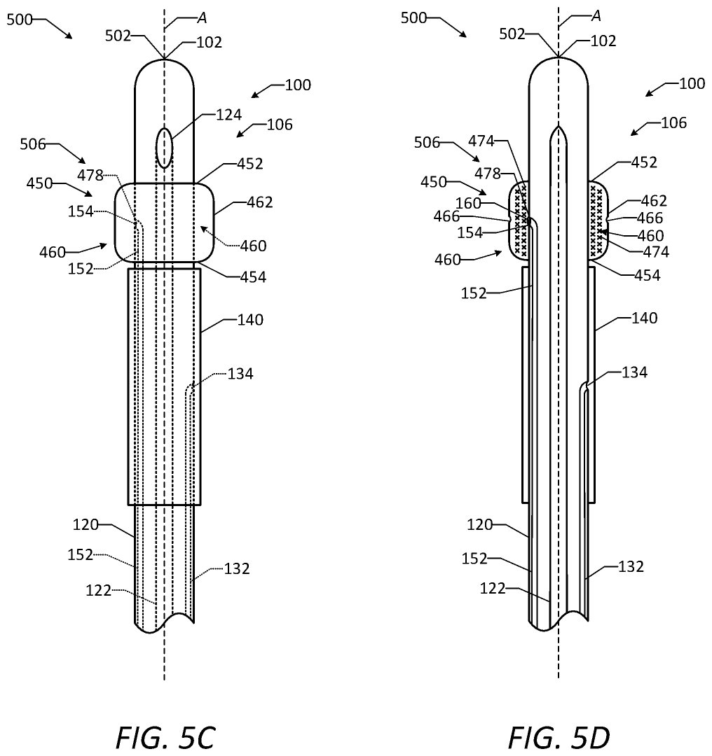

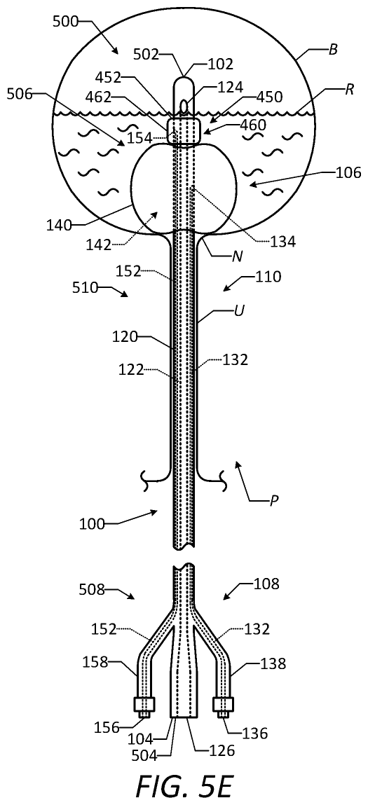

FIGS. 5A-5E illustrate a urinary catheter and drug delivery system 500 (which also may be referred to as simply a “system”) in accordance with one or more embodiments of the disclosure. As shown, the system 500 includes the urinary catheter 100, which may provide the functions described above, and the drug delivery device 450 attached to the urinary catheter 100. The drug delivery device 450 may be permanently or removably attached to the urinary catheter 100. In this manner, the urinary catheter 100 may serve as a support structure for positioning and supporting the drug delivery device 450 relative to the patient for drug delivery. Ultimately, the urinary catheter and drug delivery system 500 may allow for continuous urine drainage in addition to controlled delivery of a drug to a selected region of the patient's lower urinary tract, such as the bladder, over an extended period of time.

As shown in FIG. 5A, the urinary catheter and drug delivery system 500 has an elongated shape including a distal end 502 (which also may be referred to as a “bladder end”) and a proximal end 504 (which also may be referred to as an “external end”) positioned along a longitudinal axis A of the system 500. The system 500 includes a distal end portion 506 (which also may be referred to as a “bladder end portion”) extending from the distal end 502 toward the proximal end 504 along the longitudinal axis A, a proximal end portion 508 (which also may be referred to as an “external end portion”) extending from the proximal end 504 toward the distal end 502 along the longitudinal axis A, and an intermediate portion 510 (which also may be referred to as a “urethral portion”) extending axially from the distal end portion 506 to the proximal end portion 508. As shown, the distal end portion 506 includes the distal end portion 106 of the urinary catheter 100 and the drug delivery device 450, the proximal end portion 508 includes the proximal end portion 108 of the catheter 100, and the intermediate portion 510 includes the intermediate portion 110 of the catheter 100. When the urinary catheter and drug delivery system 500 is used for urine drainage from the a patient's bladder and drug delivery to the bladder, the distal end portion 506 may be inserted through the urethra and into the bladder, while the intermediate portion 510 resides within the urethra and the proximal end portion 508 resides outside of the patient's body.

The urinary catheter 100 illustrated in FIGS. 5A-5E is generally similar to the catheter 100 described above with respect to FIGS. 1A-1E but may include additional features described herein below. According to the embodiment of the urinary catheter 100 illustrated in FIGS. 5A-5E, the body 120 includes a drug delivery lumen 152 (which also may be referred to as a “tertiary lumen”) extending axially through the catheter 100 and configured to allow a drug or drug solution to be delivered therethrough from a drug source attached to the proximal end 104 of the catheter 100. In particular, the drug delivery lumen 152 may extend from a distal opening 154 (which also may be referred to as a “drug exit opening”) defined in the body 120 to a proximal opening 156 (which also may be referred to as a “drug entry opening”) defined in the body 120. As shown, the distal opening 154 may be defined in a sidewall of the body 120 and positioned adjacent the drug delivery device 450, and the proximal opening 156 may be defined in or near the proximal end 104 of the catheter 100. For example, the proximal opening 156 may be defined in the proximal end of a drug delivery arm 158 of the body 120. In some embodiments, as shown, the drug delivery lumen 152 has a cylindrical shape and a circular axial cross-sectional shape, although other shapes of the drug delivery lumen 152 may be used. In some embodiments, as shown, a longitudinal axis of the drug delivery lumen 152 is offset from the longitudinal axis of the body 120 and the longitudinal axis A of the system 500. In some embodiments, as shown, a valve 160 is positioned within the distal opening 154, between the drug delivery lumen 152 and the drug delivery device 450.

The drug delivery device 450 illustrated in FIGS. 5A-5E is generally similar to the drug delivery device 450 described above with respect to FIGS. 4A-4E but may include additional features described herein below. According to the embodiment of the drug delivery device 450 illustrated in FIGS. 5A-5E, the housing 462 of the drug reservoir 460 includes an opening 478 positioned adjacent the distal opening 154 of the drug delivery lumen 152. In this manner, the drug delivery lumen 152 of the catheter 100 may be in fluid communication with the drug chamber 464 of the drug reservoir 460, although such fluid communication may be controlled by the valve 160 in some embodiments. In particular, the valve 160 may be a one-way valve configured to allow fluid to flow from the drug delivery lumen 152 into the drug chamber 464 but to prevent fluid from flowing from the drug chamber 464 into the drug delivery lumen 152.

In some embodiments in which the drug 474 is not pre-loaded within the drug chamber 464, the drug delivery lumen 152 may be used to fill the drug chamber 464 with the drug 474 (in a liquid form) prior to use of the system 500 (i.e., prior to insertion of the distal end portion 506 of the system 500 through the patient's urethra and into the bladder). According to this approach, a clinician may choose to load the drug delivery device 450 with a particular drug formulation just prior to use of the system 500. In other embodiments in which the drug 474 is not pre-loaded within the drug chamber 464, the drug delivery lumen 152 may be used to fill the drug chamber 464 with the drug 474 (in a liquid form) after insertion of the distal end portion 506 of the system 500 through the patient's urethra and into the bladder. According to this approach, the reduced volume of the drug delivery device 450 (i.e., when the drug chamber 464 is empty) may ease insertion of the distal end portion 506 through the urethra and into the bladder. In these embodiments and others in which the drug 474 is pre-loaded within the drug chamber 464, the drug delivery lumen 152 also may be used to refill the drug chamber 464 with additional drug 474 (in a liquid form) after depletion of the initial drug payload.

FIG. 5E illustrates use of the urinary catheter and drug delivery system 500 to allow urine to drain from the bladder B of a patient P and also deliver a drug into the bladder B. With the drug delivery device 450 attached, either permanently or removably, to the urinary catheter 100 and the balloon 140 in the collapsed configuration, the distal end portion 506 of the system 500 may be inserted through the urethra U and into the bladder B. In particular, distal end portion 506 of the system 500 may be inserted such that the balloon 140 is disposed within the bladder B adjacent the bladder neck N, while the intermediate portion 510 of the system 500 is disposed within the urethra U and the proximal end portion 508 of the system 500 is disposed outside of the body of the patient P. Fluid, such as sterile water, then may be passed through the inflation lumen 132 and into the cavity 142 to inflate the balloon 140 to the expanded configuration, such that the balloon 140 forms a seal against the bladder neck N.

In some embodiments, the drug chamber 464 may be filled with the drug 474 either before or after insertion of the distal end portion 506 of the system 500 through the urethra U and into the bladder B. In particular, a fluid source, such as a syringe or a pump, may be attached to the proximal opening 156 of the drug delivery lumen 152 and used to deliver the drug 474 through the drug delivery lumen 152 and into the drug chamber 464. In other embodiments, the drug chamber 464 may be pre-loaded with the drug 474 during manufacture of the system 500.

With the catheter 100 positioned as shown in FIG. 5E, urine may freely enter the distal opening 124 of the drainage lumen 122, pass through the drainage lumen 122, and be collected in a collection bag attached to the proximal end 104 of the catheter 100. As shown, some residual urine R may remain in the bladder B due to the position of the distal opening 124 of the drainage lumen 122 relative to the bladder neck N With the drug delivery device 450 positioned between the distal opening 124 of the drainage lumen 122 and the balloon 140, as shown in FIG. 5E, the device 450 may reside within the residual urine R. In embodiments in which the housing 462 is formed of a water permeable material, some of the residual urine R may permeate through the wall or walls of the housing 462 and solubilize the drug 474 within the drug chamber 464. Ultimately, the drug delivery device 450 may controllably release the drug 474 into the bladder B via one or more apertures 466 or pores or through the wall or walls of the housing 462, according to one or more of the release mechanisms described above. In particular, the drug delivery device 450 may release the drug 474 directly into the bladder B. The drug 474 may be released from the drug delivery device 450 and into the bladder B over an extended period of time, such as multiple days, weeks, or months, depending on the drug payload of the drug reservoir 460.

If the drug 474 present in the drug chamber 464 becomes depleted, the drug chamber 464 may be refilled. In particular, a fluid source, such as a syringe or a pump, may be attached to the proximal opening 156 of the drug delivery lumen 152 and used to deliver new drug 474 through the drug delivery lumen 152 and into the drug chamber 464. The one-way valve 160 may maintain the new drug 474 within the drug chamber 464, preventing the new drug 474 from flowing back into the drug delivery lumen 152. The drug delivery lumen 152 advantageously may allow the drug chamber 464 to be refilled as many times as necessary to provide continued drug delivery over a desired treatment period.

Publications cited herein and the materials for which they are cited are specifically incorporated by reference. Modifications and variations of the devices, systems, and methods described herein will be obvious to those skilled in the art from the foregoing detailed description. Such modifications and variations are intended to come within the scope of the appended claims.

1. A drug delivery device for use with a urinary catheter, the drug delivery device comprising:
a drug reservoir configured to be disposed outside of a patient's body, the drug reservoir comprising:
a drug chamber containing a drug therein;
a fluid chamber containing a fluid therein; and
an osmotic barrier separating the drug chamber and the fluid chamber; and
a flexible elongate body attached to the drug reservoir and configured to traverse the patient's urethra to reach the bladder, the body comprising a drug delivery lumen extending therethrough and in fluid communication with the drug chamber.

2. The drug delivery device of claim 1, wherein the drug reservoir further comprises a housing, wherein the drug chamber is defined by a portion of the housing and the osmotic barrier, and wherein the fluid chamber is defined by a portion of the housing and the osmotic barrier.

3. The drug delivery device of claim 1, wherein the drug is in a solid form.

4. The drug delivery device of claim 3, wherein the drug is in the form of tablets, pellets, beads, granules, or powder.

5. The drug delivery device of claim 1, wherein the drug comprises an antimicrobial agent, an antifibrotic agent, an antimuscarinic agent, an anesthetic agent, an antinociceptive agent, or a combination thereof.

6. The drug delivery device of claim 1, wherein the drug comprises an agent which dissolves stones or breaks down biofilms.

7. The drug delivery device of claim 1, wherein the fluid substantially fills the fluid chamber.

8. The drug delivery device of claim 1, wherein the fluid comprises water.

9. The drug delivery device of claim 1, wherein the osmotic barrier comprises a semi-permeable wall.

10. The drug delivery device of claim 1, wherein the osmotic barrier is formed of a semi-permeable polymeric material which allows the fluid to pass therethrough but prevents the drug from passing therethrough.

11. The drug delivery device of claim 1, wherein the drug reservoir is configured to operate as an osmotic pump to drive the drug out of the drug chamber and through the drug delivery lumen.

12. The drug delivery device of claim 1, wherein the body is permanently attached to the drug reservoir.

13. The drug delivery device of claim 1, wherein the body has a tubular shape.

14. The drug delivery device of claim 1, wherein the body comprises a capillary tube.

15. The drug delivery device of claim 1, further comprising one or more releasable fasteners configured to removably attach the drug delivery device to the urinary catheter.

16. The drug delivery device of claim 1, wherein the urinary catheter is a Foley catheter.

17. A urinary catheter and drug delivery system comprising:
a urinary catheter configured to allow urine to drain from a patient's bladder, the urinary catheter comprising a flexible elongate catheter body configured to traverse the patient's urethra to reach the bladder, the catheter body comprising a drainage lumen extending therethrough; and
a drug delivery device configured for use with the urinary catheter, the drug delivery device comprising:
a drug reservoir configured to be disposed outside of the patient's body, the drug reservoir comprising:
a drug chamber containing a drug therein;
a fluid chamber containing a fluid therein; and
an osmotic barrier separating the drug chamber and the fluid chamber; and
a flexible elongate device body attached to the drug reservoir and configured to traverse the patient's urethra to reach the bladder, the device body comprising a drug delivery lumen extending therethrough and in fluid communication with the drug chamber.

18. The urinary catheter and drug delivery system of claim 17, wherein the urinary catheter is a Foley catheter.

19. The urinary catheter and drug delivery system of claim 17, wherein the urinary catheter further comprises a balloon attached to the catheter body and comprising an internal cavity, and wherein the catheter body further comprises an inflation lumen extending therethrough and in fluid communication with the internal cavity.

20. The urinary catheter and drug delivery system of claim 19, wherein the drug delivery lumen extends between catheter body and the balloon.

21. The urinary catheter and drug delivery system of claim 17, wherein the drug delivery device is attached to the urinary catheter.

22. The urinary catheter and drug delivery system of claim 21, wherein the drug delivery device is permanently attached to the urinary catheter.

23. The urinary catheter and drug delivery system of claim 21, wherein the drug delivery device is removably attached to the urinary catheter.

24. The urinary catheter and drug delivery system of claim 23, wherein the drug delivery device is removably attached to the urinary catheter by one or more releasable fasteners.

25. The urinary catheter and drug delivery system of claim 17, wherein the device body extends along an external surface of the catheter body.

26. The urinary catheter and drug delivery system of claim 25, wherein the device body is attached to the external surface of the catheter body.

27. The urinary catheter and drug delivery system of claim 17, wherein the drainage lumen extends from a distal opening to a proximal opening of the catheter body, and wherein the drug delivery lumen extends from a distal opening to a proximal opening of the device body.

28. The urinary catheter and drug delivery system of claim 27, wherein the distal opening of the drug delivery lumen is distally spaced apart from the distal opening of the drainage lumen.

29. The urinary catheter and drug delivery system of claim 28, wherein the distal opening of the drug delivery lumen is adjacent the distal opening of the drainage lumen.

30. The urinary catheter and drug delivery system of claim 17, wherein the device body extends through the drainage lumen.

31. The urinary catheter and drug delivery system of claim 17, wherein the drug is in the form of tablets, pellets, beads, granules, or powder.

32. The urinary catheter and drug delivery system of claim 17, wherein the drug substantially fills the drug chamber.

33. The urinary catheter and drug delivery system of claim 17, wherein the drug comprises an antimicrobial agent, an antifibrotic agent, an antimuscarinic agent, an anesthetic agent, an antinociceptive agent, or a combination thereof.

34. The urinary catheter and drug delivery system of claim 17, wherein the drug comprises an agent which dissolves stones or breaks down biofilms.

35. The urinary catheter and drug delivery system of claim 17, wherein the fluid substantially fills the fluid chamber.

36. The urinary catheter and drug delivery system of claim 17, wherein the fluid comprises water.

37. The urinary catheter and drug delivery system of claim 17, wherein the osmotic barrier comprises a semi-permeable wall.

38. The urinary catheter and drug delivery system of claim 17, wherein the osmotic barrier is formed of a semi-permeable polymeric material which allows the fluid to pass therethrough but prevents the drug from passing therethrough.

39. The urinary catheter and drug delivery system of claim 17, wherein the drug reservoir is configured to operate as an osmotic pump to drive the drug out of the drug chamber and through the drug delivery lumen.

40. The urinary catheter and drug delivery system of claim 17, wherein the device body is permanently attached to the drug reservoir.

41. The urinary catheter and drug delivery system of claim 17, wherein the device body has a tubular shape.

42. The urinary catheter and drug delivery system of claim 17, wherein the device body comprises a capillary tube.

43. A method of administering a drug to a patient in need thereof, the method comprising:
inserting distal end portions of a drug delivery device and a urinary catheter through the patient's urethra and positioning the distal end portions within the bladder, while maintaining proximal end portions of the drug delivery device and the urinary catheter positioned outside of the patient's body;
allowing urine to drain from the bladder through the urinary catheter; and
delivering a drug, via osmotic pressure, from the proximal end portion of the drug delivery device into the bladder.

44-61. (canceled)

62. A urinary catheter and drug delivery system comprising:
a urinary catheter configured to allow urine to drain from a patient's bladder, the urinary catheter comprising a flexible elongate catheter body configured to traverse the patient's urethra to reach the bladder, the catheter body comprising a drainage lumen extending therethrough from a distal opening to proximal opening defined in the catheter body; and
a drug delivery device attached to the urinary catheter, the drug delivery device comprising a drug reservoir positioned near the distal opening of the drainage lumen and configured to be disposed within the patient's bladder, the drug reservoir comprising a drug chamber containing a drug therein.

63-84. (canceled)

85. A method of administering a drug to a patient in need thereof, the method comprising:
inserting a drug delivery device and a distal end portion of a urinary catheter through the patient's urethra and positioning the drug delivery device and the distal end portion of the urinary catheter within the bladder, wherein the urinary catheter comprises a flexible elongate catheter body comprising a drainage lumen extending therethrough from a distal opening to proximal opening defined in the catheter body, and wherein the drug delivery device comprises a drug reservoir positioned near the distal opening of the drainage lumen and comprising a drug chamber containing a drug therein;
allowing urine to drain from the bladder through the drainage lumen; and
delivering the drug from the drug chamber into the bladder.

86-98. (canceled)
